(19) United States
(12) Patent Application Publication
(10) Pub. No.: US 2011/0004148 A1
(43) Pub. Date: Jan. 6, 2011

(54) DEVICE FOR LOCAL INTRALUMINAL TRANSPORT OF A BIOLOGICALLY AND PHYSIOLOGICALLY ACTIVE AGENT

(75) Inventor: Naoki Ishii, Isehara-shi (JP)

Correspondence Address: FOLEY & LARDNER LLP, 975 PAGE MILL ROAD, PALO ALTO, CA, 94304, US

(73) Assignee: TERUMO KABUSHIKI KAISHA

(21) Appl. No.: 12/865,851

(22) PCT Filed: Feb. 6, 2009
(86) PCT No.: PCT/US09/33482

§ 371 Date: Sep. 15, 2010

Related U.S. Application Data
(60) Provisional application No. 61/027,367, filed on Feb. 8, 2008.

(51) Int. Cl. A61N 1/30

(52) U.S. Cl. 604/20

(57) ABSTRACT

Provided herein is a drug delivery device and the method of us for the intraluminal controlled delivery of a biologically active agent comprising a dilating member comprising a proximal end and a distal end, and an inner surface and an outer surface, wherein a part of the outer surface of the dilating member is coated with a gold surface layer; a biodegradable substrate comprising the biologically active agent, wherein the substrate is covalently bonded to the gold surface layer by a gold-sulfur (Au—S—) bond; an electrical lead having a first end and a second end, the first end connected to the gold surface layer, wherein the electrical lead is configured to pass an electrical current to the gold surface layer; and wherein the controlled delivery and release of the sub strate comprising the biologically active agent is initiated by an electrical current reduction and cleavage of the Au—S bond.

### TECHNICAL FIELD

The present invention generally relates to a device for local intraluminal transport of a biologically and physiologically active agent, and more specifically, relates to a device to be inserted intralumenally into the body, e.g., via a blood vessel, for local therapeutic release of a biologically and physiologically active agent.

### BACKGROUND DISCUSSION

Drug-eluting stents are in wide use for treatment of blood vessel stenosis and the like. A drug-eluting stent dilates the blood vessel stenosis and also releases a small amount of a drug that prevents restenosis, as the stent has a surface coated with the drug for this purpose. However, drug-eluting stents are known to cause late thrombosis, and patients in whom such a stent has been implanted must take dual anti-platelet regime for prolonged periods. Attention has also focused recently on vulnerable plaque forming in less nonstenosed blood vessels as a cause of sudden death and blood vessel total occlusion. Since vulnerable plaque is not stenosis, treatment with a stent is not appropriate, and local therapeutic drug administration by catheter has been studied; but there is a problem in that the desired efficacy cannot be obtained, due to shortage of drug release period, the loss of the drug into the bloodstream and the trauma while the catheter is being introduced into the patient. This process results in the lost of the drug in the blood stream when the drug is released inside the patient.

### SUMMARY

According to one embodiment of the present application, there is provided a device for local intraluminal transport of a biologically and physiologically active agent comprising an insertion member, elongated for inserting to a lumen; a dilating member that is formed at the distal portion of the insertion member, and is radially dilatable; and a layer comprising extremely thin gold (Au) on at least part of an outer surface of the dilating member, a substance comprising a biologically and physiologically active agent being bonded to at least part of the surface via a covalent bond (Au—S—) between an SH group and the Au layer; and in addition having an electrode that is electrically connected to the layer, and an electrical line that is connected to the electrode, extending to the proximal side of the insertion member; and wherein, the substance bonded by the covalent bond is released while said dilating member has dilated and closely contacted to an inner surface of said lumen as a result of cleavage of the Au—S covalent bond by electrical power to the electrical line.

### ASPECTS OF THE INVENTION

In one embodiment, there is provided a device for local intraluminal transport and delivery of a biologically and physiologically active agent comprising: an insertion member, elongated for inserting to a lumen; a dilating member that is formed at the distally side of the insertion member, and is radially dilatable; a layer comprising extremely thin gold (Au) on at least part of an outer surface of the dilating member, a substance comprising a biologically and physiologically active agent being bonded to at least part of said surface via a covalent bond (Au—S—) between an SH group and the Au layer; an electrode that is electrically connected to said layer; and an electrical line that is connected to said electrode, extending to the proximal side of said insertion member, wherein the substance bonded by the covalent bond is released while said dilating member has dilated and closely contacted to an inner surface of said lumen as a result of cleavage of the Au—S covalent bond by electrical power to said electrical line. In another embodiment, there is provided a device for local intraluminal transport of a biologically and physiologically active agent according to the above embodiment, wherein the biologically and physiologically active agent is at least one from among: drug(s), cell(s), genes and protein. In one variation, there is provided the device for local intraluminal transport of a biologically and physiologically active agent according to the above, wherein said biologically and physiologically active agent is present on the outer surface of the dilating member in a nano- or microgranulated state, together with a biodegradable material. In one variation, the device for local intraluminal transport of a biologically and physiologically active agent according to the above, wherein said biologically and physiologically active agent is present on the outer surface of the dilating member in a microgranulated state, together with a nonbiodegradable material. In another variation, there is provided a device for local intraluminal transport of a biologically and physiologically active agent according to the above embodiment, wherein said dilating member is a balloon. In another variation, the device for local intraluminal transport of a biologically and physiologically active agent according to the above embodiment, wherein said dilating member comprises a shape memory alloy. In another variation, the device for local intraluminal transport of a biologically and physiologically active agent according to the above embodiment, wherein said dilating member is deflatable and removable from inside the body after release of said biologically and physiologically active agent.

In one embodiment, there is provided a drug delivery device for the intraluminal controlled delivery of a biologically active agent comprising: a dilating member comprising a proximal end and a distal end, and an inner surface and an outer surface, wherein a part of the outer surface of the dilating member is coated with a gold surface layer; a biodegradable substrate comprising the biologically active agent, wherein the substrate is covalently bonded to the gold surface layer by a gold-sulfur (Au—S—) bond; an electrical lead having a first end and a second end, the first end connected to the gold surface layer, wherein the electrical lead is configured to pass an electrical current to the gold surface layer; and wherein the controlled delivery and release of the substrate comprising the biologically active agent is initiated by an electrical current reduction and cleavage of the Au—S bond. In certain aspects, the substrate is non-biodegradable.

In another embodiment, there is provided a drug delivery device for the intraluminal controlled delivery of a biologically active agent comprising: an elongated insertion member having a proximal end and a distal end; a dilating member comprising a proximal end and a distal end, and an inner surface and an outer surface, wherein the dilating member is attached to the distal end of the elongated insertion member, and wherein a part of the outer surface of the dilating member is coated with a gold surface layer; a biodegradable substrate comprising the biologically active agent, wherein the substrate is covalently bonded to the gold surface layer by a gold-sulfur (Au—S—) bond; an electrical lead having a first end and a second end, the first end connected to the gold surface layer, wherein the electrical lead is configured to pass an electrical current to the gold surface layer; and wherein the controlled delivery and release of the substrate comprising the biologically active agent is initiated by an electrical current reduction and cleavage of the Au—S bond.

In another embodiment, there is provided a drug delivery device for the intraluminal controlled delivery of a biologically active agent to an intraluminal surface comprising: an elongated insertion member having a proximal end and a distal end; a dilating member comprising a proximal end and a distal end, and an inner surface and an outer surface, wherein the dilating member is attached to the distal end of the elongated insertion member, and wherein a part of the outer surface of the dilating member is coated with a gold surface layer; a biodegradable substrate comprising the biologically active agent, wherein the substrate is covalently bonded to the gold surface layer by a gold-sulfur (Au—S—) bond; a first electrical lead having a first end and a second end, the first end connected to the gold surface layer, wherein the first electrical lead is configured to pass an electrical current to the gold surface layer; and a second electrical lead having a first end and a second end, the first end connected to a counter electrode, wherein the second electrical lead is configured to pass an electrical current to the counter electrode; wherein the controlled delivery and release of the substrate comprising the biologically active agent is initiated when the dilating member is directly contacting to the intraluminal surface, and is initiated by an electrical current reduction and cleavage of the Au—S bond. In one variation, the gold surface layer is placed only on the portion of the dilating member that in direct contact with the intraluminal surface when the dilating member is dilated; and at least a part of the counter electrode is placed on a portion that is not directly in contact with the intraluminal surface when the dilating member is dilated; the second end of the first electrical lead connected to the anode at the proximal side; the second end of the second electrical lead connected to the cathode at the proximal side, wherein the controlled delivery and release of the substrate comprising the biologically active agent is initiated when the dilating member is directly contacting to the intraluminal surface is initiated by an electrical current reduction from the proximal side of the device and cleavage of the Au—S bond. In another variation of the above device, the dilating member is a coronary scaffold or a balloon. In another variation, the outer surface of the coronary scaffold or the balloon is coated with the gold surface layer. In another variation, the counter electrode main body on the balloon is placed on a proximal corn part of the balloon that does not directly contact the intraluminal surface when the balloon is dilated. In another variation, the junction of the electrical leads connected to the gold surface layer and the counter electrode on the balloon is covered with an outer shaft material of the elongated insertion member or an miscible materials with the outer shaft materials. In another variation, the counter electrode is placed in a distal portion of elongated insertion member. In yet another variation, at least an insulation layer is configured from the proximal to the distal of the elongated insertion member to separate the first electrical lead from the second electrical lead. In another variation of the above devices, a ratio of the surface area of the gold surface layer on the dilating member/all surface area of counter electrodes is not less than 1. In a particular variation of the above device, the dilating member is a coronary scaffold or a balloon. In another variation, the outer surface of the coronary scaffold or the balloon is coated with the gold surface layer. In yet another variation, the dilating member is a balloon and no portion of the gold surface layer exists on a folding line of the balloon. In another variation of the device, the surface area of the gold surface layer is more than at least about 20% of a surface area of the dilating member contacting an intraluminal surface. In a particular variation of the above, a surface area of the biodegradable substrate is more than at least about 20% of the surface area of the gold surface layer. In yet another variation of the device, the device further comprises a second electrical lead having a first end and a second end, the first end connected to a counter electrode. In a further variation, a portion of the counter electrode directly contacts a body fluid. In yet another variation of the device, the shortest distance between the gold surface layer and the counter electrode is 0.01 mm-100 mm.

In another variation, the first and second electrical leads are covered with an insulation layer. In a particular variation, the coronary scaffold is made from a metal selected from the group consisting of stainless steel, platinum, titanium, tantalum, nickel-titanium, cobalt-chromium and their alloys thereof, or is made from a shape memory alloy or a superelastic alloy is selected from the group consisting of copper-zinc-aluminum-nickel, copper-aluminum-manganese, copper-aluminum-nickel and nickel-titanium alloy. In yet another variation, the gold surface layer has a thickness of between 0.05 micron and 50 microns. In another variation, the gold surface layer is about 0.05 microns, or about 50 microns, or between 0.1 and 20 microns, or between 0.1 and 10 microns.

In another variation of the above device, the biodegradable substrate comprising a sulfur atom is covalently bonded to a hydrophobic fragment and a hydrophilic fragment, wherein the hydrophobic fragment comprises a biologically active agent; or wherein the biodegradable substrate comprising a sulfur atom is covalently bonded to a hydrophobic fragment that is bonded to a hydrophilic fragment that is further bonded to a hydrophobic fragment, wherein the hydrophobic fragment comprises a biologically active agent. In one variation of the above, the hydrophobic fragment is a —C₅₋₁₈alkylenyl- and the linker is selected from the group consisting of —C(O)O—, —C(O)NH—, —OC(O)O—, —OC(S)O—, —OC(O)NH—, —NR¹C(O)O—, —SC(O)O—, —SC(O)S—, —NR¹C(NR¹)O— and —NR¹C(O)NR¹—, wherein each R¹ is independently H or C₁₋₃alkyl. In another variation, the hydrophilic fragment comprises a biodegradable polymer selected from the group consisting of PAE, PCL, PLLA, PLA, PLGA, PHB, POE, polyketal, polyanhydride, polypeptide and PAE, and wherein the end group is selected from the group consisting of —OH, —NH₂, —C(O)OH, —NCO, —SH, biotin, and their block copolymer combinations thereof. In one aspect, the particular polymers that may be employed include PAE (poly amide ester), PCL (poly(ε-caprolactone)), PLLA (Poly-(L-lactide)), PGA (polyglycolic acid or polyglycolide), PLA (poly(D, L-lactic acid) and polylactide), PHB (poly hydroxybutyrate), POE (poly ortho ester), polyketal, polyanhydride, polypeptide, PAE (poly(β-amino ester)), and combinations thereof. In one variation of the above, the hydrophilic fragment comprises a biodegradable polymer that forms nanoparticles, nanogranulated particles, microparticles or microgranulated particles encapsulating the biologically active agent. In one aspect, the biologically active agent may be absorbed, embedded and/or entrapped within the polymer. In another aspect, the biologically active agent is attached to the polymer by a covalent bond, non-covalent bond, a biodegradable bond, a hydrogen bond, a Van der Waals interaction or an electrostatic interaction.

In a particular variation of the above, the hydrophobic fragment and the hydrophilic fragment is —[—(C₅₋₁₈alkylenyl)ₘ-L-(CH₂CH₂O)ₙ—]ₚ—, wherein L is a linker selected from the group consisting of —C(O)O—, —C(O)NH—, —OC(O)O—, —OC(S)O—, —OC(O)NH—, —NR¹C(O)O—, —SC(O)O—, —SC(O)S—, —NR¹C(NR¹)O— and —NR¹C(O)NR¹—, wherein each R¹ is independently H or C₁₋₃alkyl, and where m is 1, 2 or 3, n is 1 to 90, and p is 1 to 10. In a particular variation of the above device, the biologically active agent is selected from the group consisting of a carcinostatic, an immunosuppressive, an antihyperlipidemic, an ACE inhibitor, a calcium antagonist, an integrin inhibitor, an antiallergic, an antioxidant, a GPIIb/IIIa antagonist, retinoid, flavonoid, carotenoid, a lipid improvement agent, a DNA synthesis inhibitor, a tyrosine kinase inhibitor, an antiplatelet, a vascular smooth muscle antiproliferative agent, an anti-inflammatory agent, a biological material, an interferon and a NO production accelerator. In one aspect, the biologically active agents are substantially water soluble agents or water soluble drugs. The biologically active agents may include antithrombotics, antiproliferatives, anti-inflammatory agents, smooth muscle cell migration inhibitors and restenosis-reducing agents. Particular biologically active agents include paclitaxel, sirolimus, simvastatin and rapamycin. In certain aspects, the total load of the biologically active agents may be about 1-1,000 μg, 1-250 μg, 1-100 μg, 1-50 μg, 1-25 μg, 1-10 μg or about 5 μg, the dose of which depends on the nature and biological activity of the agents. The calculation of the dosages are previously known to one skilled in the art. In another variation of the above device, the dilating member is a self-expandable scaffold or a shape memory scaffold. In one variation, the dilating member is circumferentially loaded with a continuous gold layer. In another variation, the dilating member is partially loaded with a continuous gold layer. In a particular variation of the above device, the dilating member is a balloon and the gold surface layer comprises discontinuous rectangle-shaped gold layers. In yet another variation, the dilating member is a balloon and the gold surface layer comprises discontinuous wave-shaped gold layers.

In another embodiment, there is provided a method for the controlled delivery of a biologically active agent to an intraluminal surface using a drug delivery device, wherein the device comprises: an elongated insertion member having a proximal end and a distal end; a dilating member comprising a proximal end and a distal end, and an inner surface and an outer surface, wherein the proximal end of the dilating member is attached to the distal end of the elongated insertion member, and wherein a part of the surface of the dilating member is coated with a gold surface layer; a biodegradable substrate comprising the biologically active agent, wherein the substrate is covalently bonded to the gold surface layer by a gold-sulfur (Au—S—) bond; an electrical lead having a first end and a second end, the first end connected to the gold surface layer, wherein the electrical lead is configured to pass an electrical current to the gold surface layer; and wherein the controlled delivery and release of the substrate comprising the biologically active agent is initiated by a electrical current reduction and cleavage of the Au—S bond; the method comprises inserting the device into the lumen and advancing the device until the dilating member is in a desired region of the intraluminal surface; expanding the dilating member to contact the outer surface of the dilating member with the vessel wall; and passing an electrical current to the electrical lead sufficient to reduce and cleave the Au—S bond and releasing the biodegradable substrate comprising the biologically active agent over a controlled time period. In one aspect, the controlled time period is between 0.1 and 120 seconds, or between 5 and 30 seconds, between 10 and 20 seconds, or between 1 and 10 seconds, between 1 and 20 seconds, between 1 and 30 seconds, or between 30 and 60 seconds, between 40 and 60 seconds or between 50 and 60 seconds. In one aspect, the release of the substrate comprising the biologically active agent from the device may be performed at low electrical currents. The electrical current are generated at biologically safe levels. The release of the substrate may be performed using electrochemically programmed methods to release the agent at the desired levels, rate. The release of the substrate may be programmed to provide the biological agent at the desired concentrations. The programmed release of the substrate from the gold surface may be biased at about −1.5 V (vs. Ag/AgCl) for the desired about of time. See “Electrochemically Programmed Release of Biomolecules and Nanoparticles, Nano Letters, ACS, vol. 6, no. 6, pp. 1250-1252 (2006), the reference of which is incorporated herein in its entirety. In a particular variation of the above method, the method further comprises a step of contracting the dilating member and withdrawing the device from the lumen. In one variation, the dilating member is a coronary scaffold or a balloon. In another variation, the region of the lumen comprises vulnerable plaque. In another variation, the biodegradable substrate comprising a sulfur atom is covalently bonded to a hydrophobic fragment and a hydrophilic fragment, wherein the hydrophobic fragment comprises a biologically active agent; or wherein the biodegradable substrate comprising a sulfur atom is covalently bonded to a hydrophobic fragment that is bonded to a hydrophilic fragment that is further bonded to a hydrophobic fragment, wherein the hydrophobic fragment comprises a biologically active agent. In a particular variation of the above method, the hydrophobic fragment is a —C₅₋₁₈alkylenyl- and the linker is selected from the group consisting of —C(O)O—, —C(O)NH—, —OC(O)O—, —OC(S)O—, —OC(O)NH—, —NR¹C(O)O—, —SC(O)O—, —SC(O)S—, —NR¹C(NR¹)O— and —NR¹C(O)NR¹—, wherein each R¹ is independently H or C₁₋₃alkyl. In another variation, the hydrophilic fragment comprises a biodegradable polymer selected from the group consisting of PAE, PCL, PLLA, PLA, PLGA, PHB, POE, polyketal, polyanhydride, polypeptide and PAE, and wherein the end group is selected from the group consisting of —OH, —NH₂, —C(O)OH, —NCO, —SH, biotin, and their block copolymer combinations thereof. The particular polymers that may be employed include PAE (poly amide ester), PCL (poly(ε-caprolactone)), PLLA (Poly-(L-lactide)), PGA (polyglycolic acid or polyglycolide), PLA (poly(D, L-lactic acid) and polylactide), PHB (poly hydroxybutyrate), POE (poly ortho ester), polyketal, polyanhydride, polypeptide, PAE (poly(β-amino ester)), and combinations thereof. In another variation of the above method, the hydrophilic fragment comprises a biodegradable polymer that forms nanoparticles, nanogrannulated particles, microparticles or microgranulated particles encapsulating the biologically active agent. In a particular variation, the hydrophobic fragment and the hydrophilic fragment is —[—(C₅₋₁₈alkylenyl)ₘ-L-(CH₂CH₂O)ₙ—]ₚ—, wherein L is a linker selected from the group consisting of —C(O)O—, —C(O)NH—, —OC(O)O—, —OC(S)O—, —OC(O)NH—, —NR¹C(O)O—, —SC(O)O—, —SC(O)S—, —NR¹C(NR¹)O— and —NR¹C(O)NR¹—, wherein each R¹ is independently H or C₁₋₃alkyl, and where m is 1, 2 or 3, n is 1 to 100, and p is 1 to 10. In certain variations, n is 1-10, n is 1-20, n is 10-30 or n is 20-50. In certain variations, the PEG has a molecular weight of about Mw 60-5,400. In another variation of the above, the biologically active agent is selected from the group consisting of a carcinostatic, an immunosuppressive, an antihyperlipidemic, an ACE inhibitor, a calcium antagonist, an integrin inhibitor, an antiallergic, an antioxidant, a GPIIb/IIIa antagonist, retinoid, flavonoid, carotenoid, a lipid improvement agent, a DNA synthesis inhibitor, a tyrosine kinase inhibitor, an antiplatelet, a vascular smooth muscle antiproliferative agent, an anti-inflammatory agent, a biological material, an interferon, and a NO production accelerator.

In another embodiment, there is provided a method of preparing a drug delivery device comprising a dilating member, with a substrate, the method comprising: coating an outer surface of the dilating member in a dilated state with a layer of gold; contacting the layer of gold with hydrophobic compound comprising a functional group and a thiol group, for a sufficient time to form a gold-sulfur (Au—S) bond between the hydrophobic compound and the layer of gold; contacting the functional group of the hydrophobic compound with an activating group for a sufficient time to form an activated hydrophobic compound; and contacting the activated hydrophobic compound with a hydrophilic polymer comprising a biologically active agent and an amine group to form the substrate. In one variation of the method, the dilating member is a coronary scaffold or a coronary balloon that is secured to a catheter. In another variation, the coating of the outer surface of the dilating member is performed by dispensing, pipetting, ink jet deposit or chemical vapor deposition. In yet another variation, the hydrophilic polymer comprising a biologically active agent forms a nano-granule, a micro-granule, a nanoparticle, or a microparticle. In a particular variation of the above method, the activated hydrophobic compound and the substrate form a self-assembled monolayer (SAM).

### BRIEF DESCRIPTION OF THE FIGURES

FIGS. 1a-1d show various views of an example of a balloon catheter embodiment having a first electrode design.

FIGS. 2a-2d show various views of an example of a balloon catheter embodiment having a second electrode design.

FIGS. 3a-3c show various views of an example of a balloon catheter embodiment having a third electrode design.

FIGS. 4a-4d show various views of an example of a balloon catheter embodiment having a fourth electrode design.

FIGS. 5a-5d show various views of an example of a balloon catheter embodiment according to a first balloon and outer shaft arrangement in which the electrical leads are embedded in the outer shaft.

FIGS. 6a-6d show various views of an example of a balloon catheter embodiment according to a first balloon and outer shaft arrangement in which the electrical leads travel along the outside of the outer shaft.

FIGS. 7a-7b show two side perspective cross-sectional views of an example of a balloon catheter embodiment according to a second balloon and outer shaft arrangement in which the electrical leads are embedded in the outer shaft.

FIGS. 8a-8b show two side perspective cross-sectional views of an example of a balloon catheter embodiment according to a second balloon and outer shaft arrangement in which the electrical leads in a helical formation along the outside of the inner shaft.

FIGS. 9a-9f show various views of examples of how the electrical leads can be fused to the gold and counter electrodes according to various examples of a balloon catheter embodiment.

FIGS. 10a-10d show various views of an example of a stent-delivery catheter embodiment in an over-the-wire system according to a first self-expandable scaffolding design.

FIGS. 11a-11d show various views of an example of a stent-delivery catheter embodiment in a rapid exchange system according to a first self-expandable scaffolding design.

FIGS. 12a-12d show various views of an example of a stent-delivery catheter embodiment in an over-the-wire system according to a second self-expandable scaffolding design.

FIGS. 13a-13b show two side perspective views of an example of a stent-delivery catheter embodiment.

FIGS. 14a-14c show various views of a shape memory scaffold that can be used in a stent-delivery catheter embodiment.

### DETAILED DESCRIPTION

As used herein, a “biologically and physiologically active agent” or “active agent” may include drugs, cells, genes and protein. In particular, non-limiting active agents may include therapeutic drugs for treating or preventing restenosis, and may include anti-platelet agents, anti-coagulant agents, anti-fibrin agents, anti-inflammatory agents, anti-thrombin agents and anti-proliferative agents. Other non-limiting active agents may include a growth factor, a statin, a toxin, an antimicrobial agent, an analgesic, an anti-metabolic agent, a vasoactive agent, a vasodilator agent, a prostaglandin, a hormone, a thrombin inhibitor, an enzyme, an oligonucleotide, a nucleic acid, an antisense, a protein, an antibody, an antigen, a vitamin, an immunoglobulin, a cytokine, a cardiovascular agent, endothelial cells, an antibiotic, a chemotherapeutic agent, an antioxidant, a phospholipid, a corticosteroid, a heparin, a heparinoid, albumin, a gamma globulin, paclitaxel, hyaluronic acid and any combination thereof.

As used herein, “linker” refers to the group L that may be selected from the group —C(O)O—, —C(O)NH—, —OC(O)O—, —OC(S)O—, —OC(O)NH—, —NR¹C(O)O—, —SC(O)O—, —SC(O)S—, —NR¹C(NR¹)O— and —NR¹C(O)NR¹— wherein each R¹ is independently H or C₁₋₃alkyl, or as defined herein. The linker is a carbonyl-based functional group (i.e., —C(O)O—, —C(O)NH—, —C(S)—, —C(O)(NR¹)— etc . . . ) that links or connects the hydrophobic fragment or the hydrophobic fragment with the hydrophilic fragment. Accordingly, depending on the particular atom (i.e., O, N or S) that the hydrophobic group and/or the hydrophilic group terminates in and connects to the linker, the oxygen, nitrogen or sulfur atom explicitly shown as comprising part of the above linker groups, may be present or may be absent. That is, the linker may also be represented as —OC(O)—, —C(O)NH—, —C(S)— and —C(O)(NR¹)—.

As used herein, a “stent” or “scaffold” (as used interchangeably herein) may be a dilating member, where the scaffold may be used in a similar manner as a PTCA procedure or balloon angiography procedure using a drug eluting balloon. The PTCA procedure using the scaffold, is performed by threading a slender balloon-tipped tube, such as a catheter, from an artery in the groin to a selected location in an artery of the heart. The scaffold is then dilated or expanded, compressing the plaque and dilating (widening) the narrowed coronary artery so that blood can flow more easily. As disclosed herein, controlled delivery of the biologically active agent may be performed using the present procedure. The scaffold may be made from a shape memory alloy. Once the procedure is completed, the scaffold may be withdrawn, along with the catheter, from the artery. The scaffold may be made in part, from a metallic material. Non-limiting examples of such metallic materials include stainless steel, platinum, titanium, tantalum, nickel-titanium, cobalt-chromium and their alloys thereof.

“Substrate” refers to a composition comprising a thiol group that bonds to the gold surface to form a sulfur-gold (S—Au) bond. The substrate may further comprise a hydrophobic linker or a hydrophobic chain, such as a C₅₋₁₈alkyl group, that is linked or attached to a hydrophilic component, such as a PEG group or a polypeptide polymer. The substrate may further comprise a biologically active agent that may be delivered during the controlled release of the substrate from the gold surface upon the cleavage of the S—Au bond.

### Catheter Devices:

The present invention may be applied to catheters and stents or any other drug delivery device system. The catheter depicted in the majority of these Figures are balloon or stent delivery catheters. However it can be appreciated that the catheter can be any one of multiple different intravascular or non-intravascular catheter types. A person of ordinary skill in the art will be familiar with different types of catheters appropriate for multiple embodiments.

In an embodiment of the invention, a balloon catheter 100 comprising a balloon 110, an inner shaft 130, an outer shaft 140, gold electrodes 150 with gold electrode leads 156, counter electrodes 160 with counter electrode leads 166, and a radiopaque marker band 165 is used in conjunction with the drug delivery system described above.

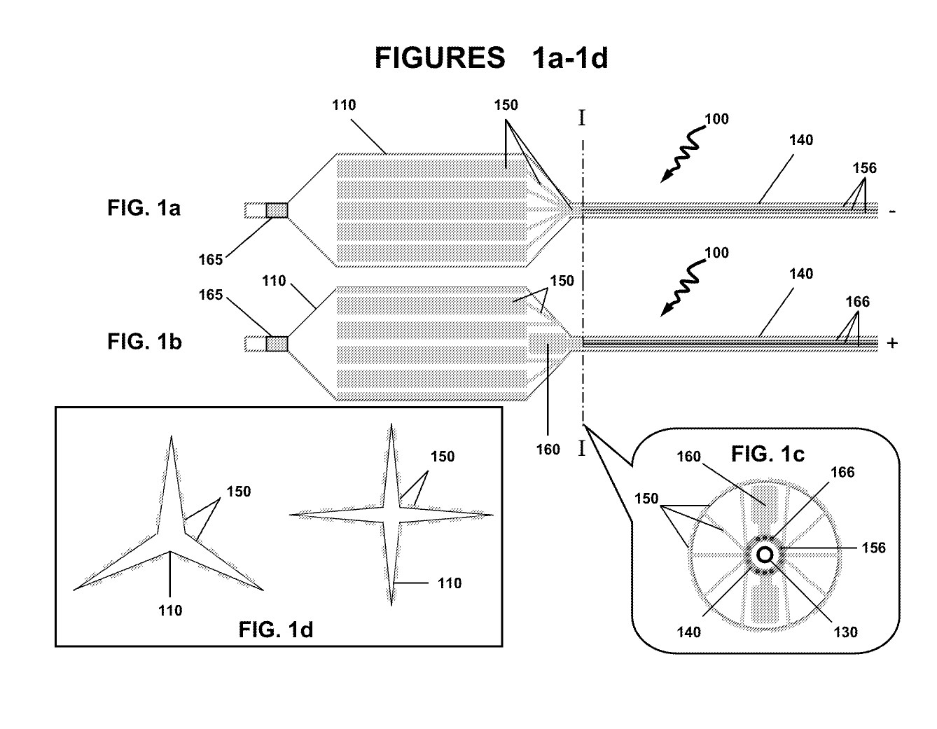

Various electrode designs may be used in a balloon catheter 100, though four are described. In all of the following electrode designs in FIGS. 1-4, the balloon 110 is shown unfolded outside of the outer shaft 140. The balloon 110 has a proximal tapered section, a non-tapered intermediate section, and a distal tapered section. Current travels from a current source near the proximal end of the catheter through the counter electrode leads 166 to counter electrodes 160, a portion of which is located on the balloon 110. Preferably, the counter electrodes 160 are located on the proximal tapered section of the balloon 110 to ensure that the counter electrodes 160 can easily contact blood or body fluid. Though in the electrode designs shown below, two counter electrodes 160 are shown on opposite sides of the outside surface of the balloon 110, it should be understood that more counter electrodes 160 (or even a single counter electrode 160) may be used. The current then travels from the counter electrodes 160 through the blood or body fluid to the gold electrodes 150 located on the balloon 110. To prevent the current from traveling into the human body, the counter electrodes 160 should be located as close to the gold electrodes 150 as possible, without actually contacting the counter electrodes 160 and gold electrodes 150 together. Preferably, the distance between the portions of the counter electrodes 160 and gold electrodes 150 nearest each other is between 0.01 mm-100 mm. The gold electrodes 150 comprise electrically conductive plates substantially made out of gold or at least layered with a thin gold film. The gold electrodes 150 are circumferentially located on the outside of the balloon 110. The gold electrodes 150 comprise the biologically active materials described above. The counter electrodes 160 are positively charged with regard to the gold electrodes 150. The gold electrode leads 156 are used to complete the circuit at the proximal end of the catheter. The electrical leads 156 and 166 should be covered with insulation as much as possible to prevent electricity leakages. The radiopaque marker band 165 is shown situated external to the distal end of the balloon 110.

In FIGS. 1a and 1b, two top perspective views of the distal end of balloon catheter 100 with a first electrode design are shown. The two top perspective views of the balloon catheter 100 are taken from perpendicular angles. The portion of these top perspective views to the right of the dotted line I-I is a cross-sectional view of the outer shaft 140 showing the electrical leads 156 and 166 traveling the length of the balloon catheter 100. FIG. 1a shows a top perspective of the balloon catheter 100 which emphasizes the arrangement of the gold electrodes 150. The gold electrodes 150 are arranged circumferentially around the outside of the balloon 110 and have a rectangular bar shape. The greater the surface area of the outside of the balloon 110 that is covered with gold electrodes 150, the greater the coverage of the drug distribution to the surrounding intraluminal surfaces. The electrodes 156 are generally placed discontinuously, but evenly, around the entire circumference of the balloon 110. Electrical leads 156 are connected to these gold electrodes 150, preferably via a solder, and are embedded inside the outer shaft 140 as they travel between the proximal and distal ends of the balloon catheter 100. FIG. 1b shows the gold electrodes 150 circumferentially placed along the outside of the balloon 110 in relation to a counter electrode 160. The counter electrode 160 is on the proximal end of the balloon 110. Counter electrode leads 166 are embedded in the outer shaft and can be seen running from the counter electrode 160 towards the proximal end of the balloon catheter 100. FIG. 1c shows an exemplary cross-sectional view of the entire catheter along the dotted line I-I in FIGS. 1a and 1b. The gold electrodes 150 are shown placed along the outside of the balloon 110. Electrical leads 156 are shown embedded in the outer shaft 140. The counter electrodes 160 are shown situated on opposite sides of the outside surface of the balloon 110. The portions of the counter electrodes on the proximal end of the outside of the balloon 110 extend towards the gold electrodes 150 on the outside of the balloon 110, but do not actually contact the gold electrodes 150. FIG. 1d shows cross-sectional views of two alternative folding patterns for the balloon 110 when it is folded inside the balloon catheter 110. One view shows the balloon 110 folded into three portions, while the other view shows the balloon 110 folded into four portions. Preferably, the gold electrodes 150 loaded with the drug delivery system described above are not placed on the creases of the folded balloon 110 in order to avoid short circuiting.

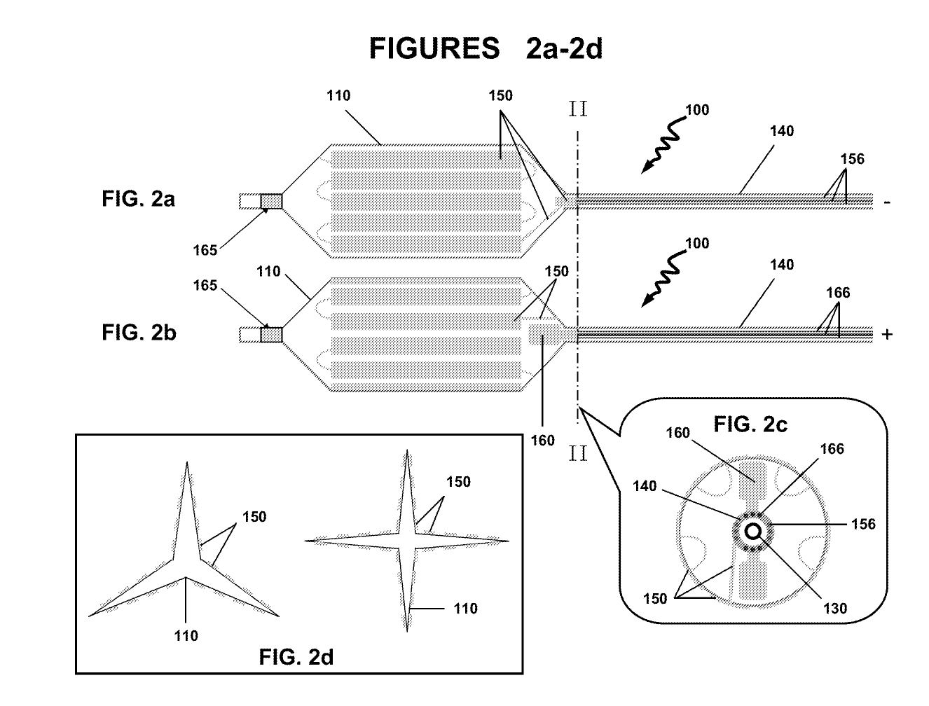

FIGS. 2a-2d show another example of the electrode design at the distal end of a balloon catheter 100 embodiment of the invention. In FIGS. 2a and 2b, two top perspective views of a balloon catheter 100 with a second electrode design are shown. The two top perspective views of the balloon catheter 100 are taken from perpendicular angles. The portion of these top perspective views to the right of the dotted line II-II is a cross-sectional view of the outer shaft 140 showing the electrical leads 156 and 166 traveling the length of the balloon catheter 100. Instead of all the gold electrodes 150 circumferentially located on the balloon 110 having portions extending into the outer shaft 140, only one gold electrode 150 has such an extending portion. This portion is electrically connected to the gold electrode leads 156. All of the gold electrodes 150 are connected to each other in series. FIG. 2c shows a cross-sectional view of the entire balloon catheter 100 along the dotted lines II-II in FIGS. 2a and 2b. FIG. 2d shows cross-sectional views of two alternative folding patterns for the balloon 110 when it is folded inside of the balloon catheter 100.

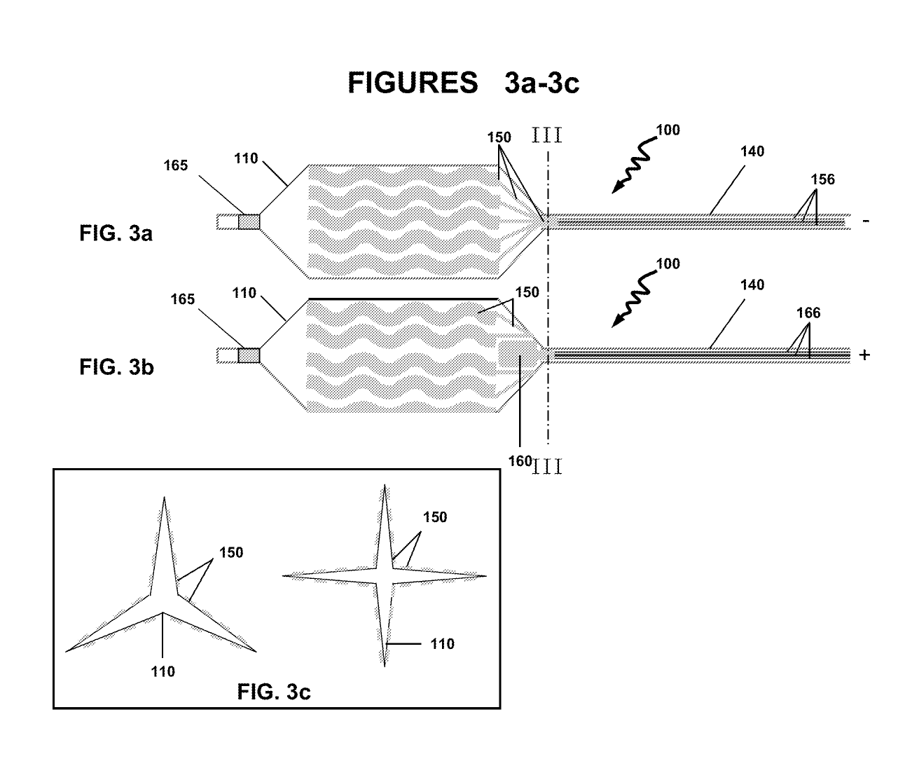

FIGS. 3a-3c show another example of the electrode design at the distal end of a balloon catheter 100 embodiment of the invention. In FIGS. 3a and 3b, two top perspective views of a balloon catheter 100 with a third electrode design are shown. The two top perspective views of the balloon catheter 100 are taken from perpendicular angles. The portion of these top perspective views to the right of the dotted line III-III is a cross-sectional view of the outer shaft 140 showing the electrical leads 156 and 166 traveling the length of the balloon catheter 100. The electrical lead system in this example is similar to the example in FIGS. 1a-1d. However, the shape of the gold electrodes 150 circumferentially placed along the outside of the balloon 110 is different. Instead of a rectangular bar shape, the gold electrodes 150 have a wavy line shape. Theses wavy-line-shaped electrodes allow the balloon surface to have greater flexibility and, thus, allow the balloon to move more smoothly through a vessel. FIG. 3c shows cross-sectional views of two alternative folding patterns for the balloon 110 when it is folded inside of the balloon catheter 100.

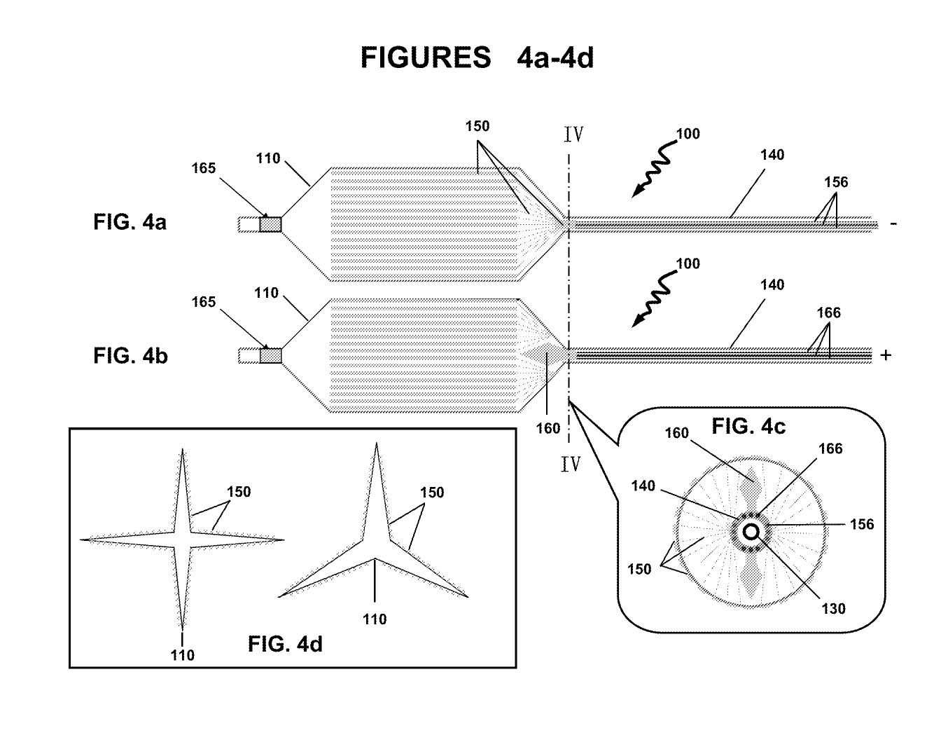

FIGS. 4a-4d show another example of the electrode design at the distal end of the balloon catheter 100 embodiment of the invention. In FIGS. 4a and 4b, two top perspective views of a balloon catheter 100 with a fourth electrode design are shown. The portion of these top perspective views to the right of the dotted line IV-IV is a cross-sectional view of the outer shaft 140 showing the electrical leads 156 and 166 traveling the length of the balloon catheter 100. The two top perspective views of the balloon catheter 100 are taken from perpendicular angles. The electrical lead system in this example is similar to the example in FIGS. 1a-1d. The rectangular-bar shaped gold electrodes 150 are less wide than the ones used in FIG. 1, and thus, more of them are placed circumferentially around the balloon 110. This results in a greater quantity of electrical leads running from the gold electrodes 150 through the walls of the outer shaft 140. FIG. 4c shows a cross-sectional view of the entire catheter along the dotted line IV-IV in FIGS. 4a and 4b. The greater number of gold electrodes 150 are clearly shown. FIG. 4d shows cross-sectional views of two alternative folding patterns for the balloon 110 when it is folded inside of the balloon catheter 100.

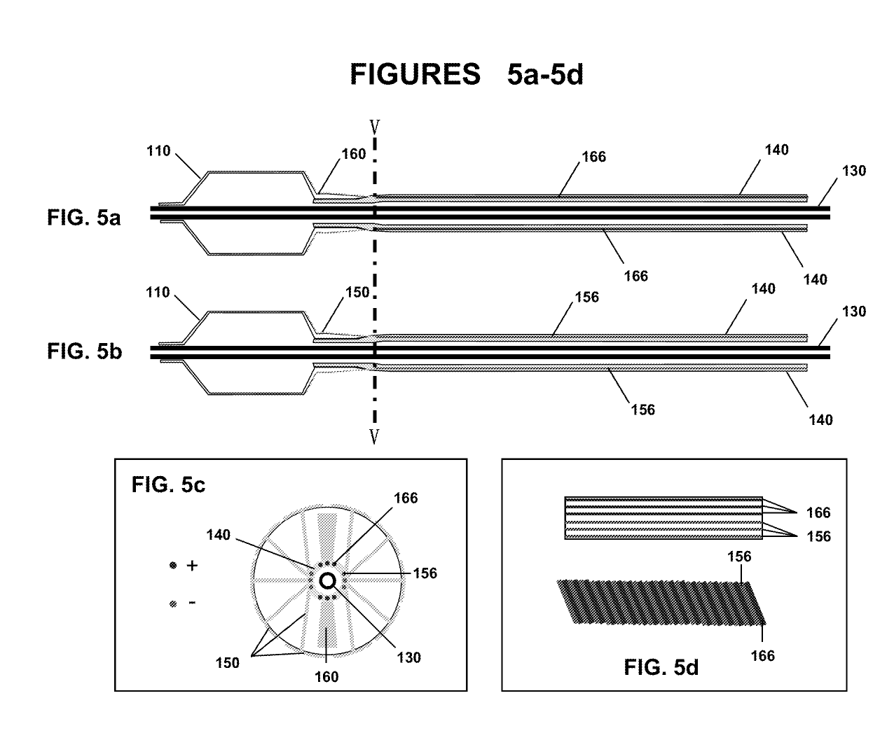

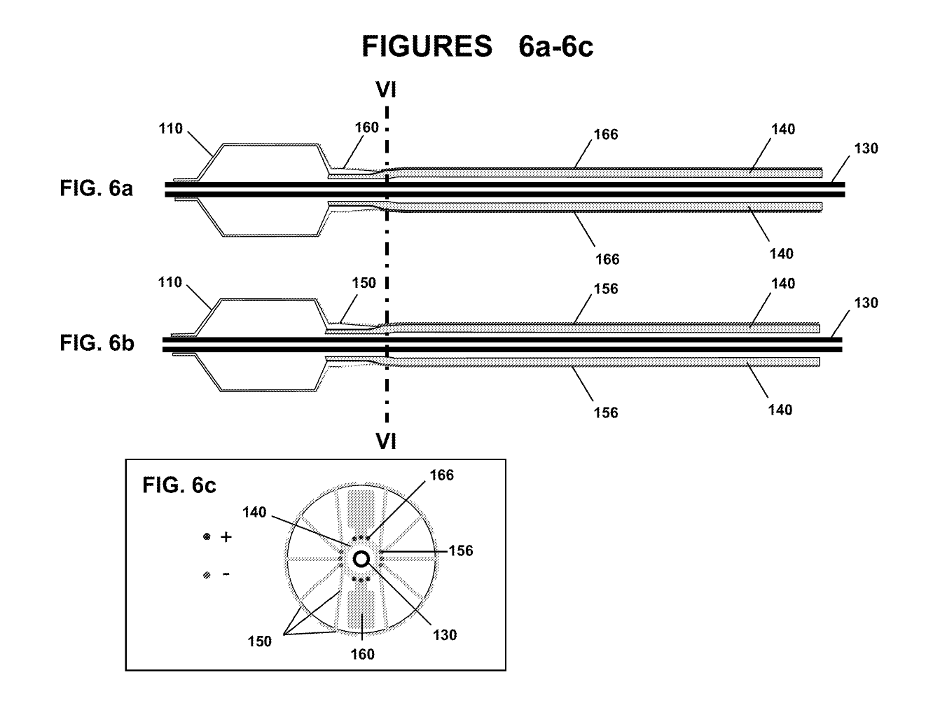

In the balloon catheter embodiment, the balloon 110 and outer shaft 140 may be connected to each other in two alternative arrangements. In a first arrangement, shown in FIGS. 5-6, the distal end of the outer shaft 140 is inserted into the proximal end of the balloon 110. This arrangement allows the catheter 100 to have a smaller profile and creates a generally smoother exterior surface at the distal end of the catheter 100. FIGS. 5a-5b are cross-sectional side views of the distal end of a balloon catheter 100 demonstrating this first arrangement. The electrical leads 156 and 166 traveling from the gold electrodes 150 and counter electrodes 160 are shown embedded in the outer shaft 140. FIG. 5c is a cross-sectional view taken along the dotted line V-V in FIGS. 5a and 5b. The counter electrodes 160 and gold electrodes 150 can be seen, in addition to the electrical leads 156 and 166 embedded in the outer shaft 140. FIG. 5d shows two views of how the electrical leads 156 and 166 embedded in the outer shaft 140 can be arranged. The electrical leads 156 and 166 are shown travelling in parallel along a linear line, or as one alternative, travelling in a helical formation.

FIGS. 6a-6b are cross-sectional views of the distal end of a balloon catheter 100 showing another example of this first arrangement. The electrical leads 156 and 166 are situated on the outside of the outer shaft 140 instead of embedded in the outer shaft 140 as was shown in FIGS. 5a-5d. FIG. 6c is a cross-sectional view taken along the dotted line VI-VI of FIGS. 6a and 6b. The counter electrodes 160 and gold electrodes 150 can be seen, in addition to the electrical leads 156 and 166 situated on the outside of the outer shaft 140. In either example of this first arrangement, the electrical leads 156 and 166 are manageable from the proximal side of the catheter.

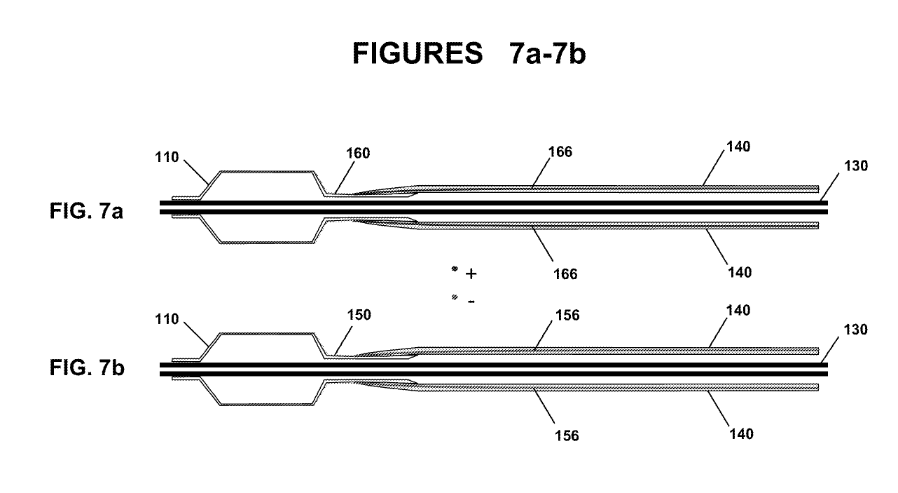

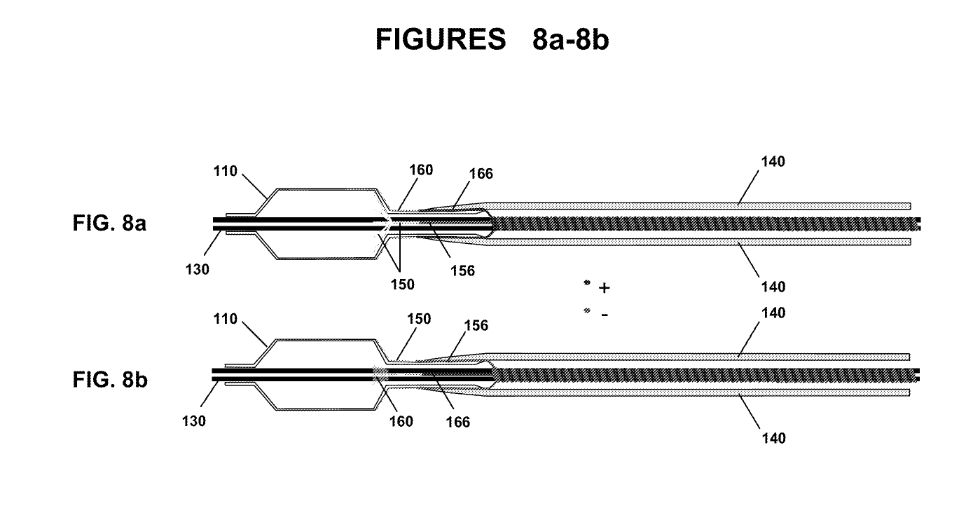

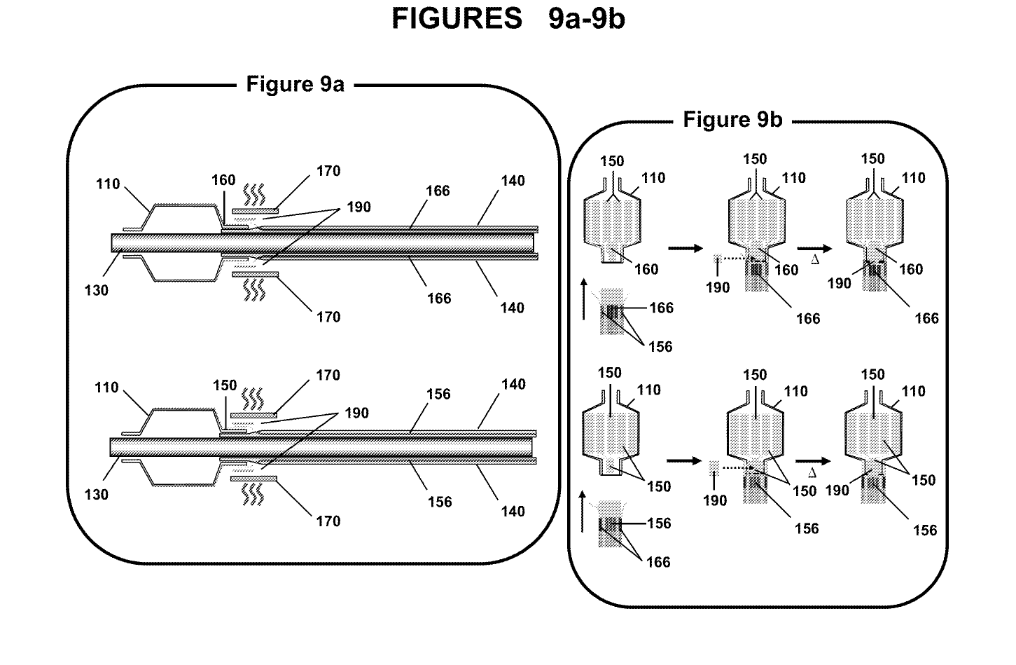

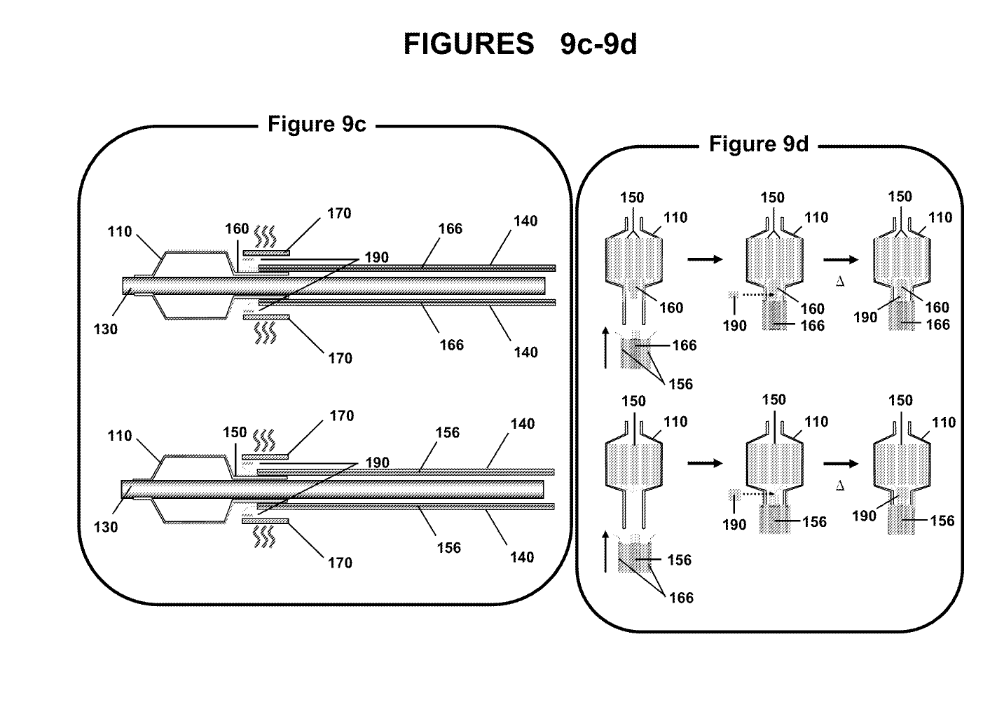

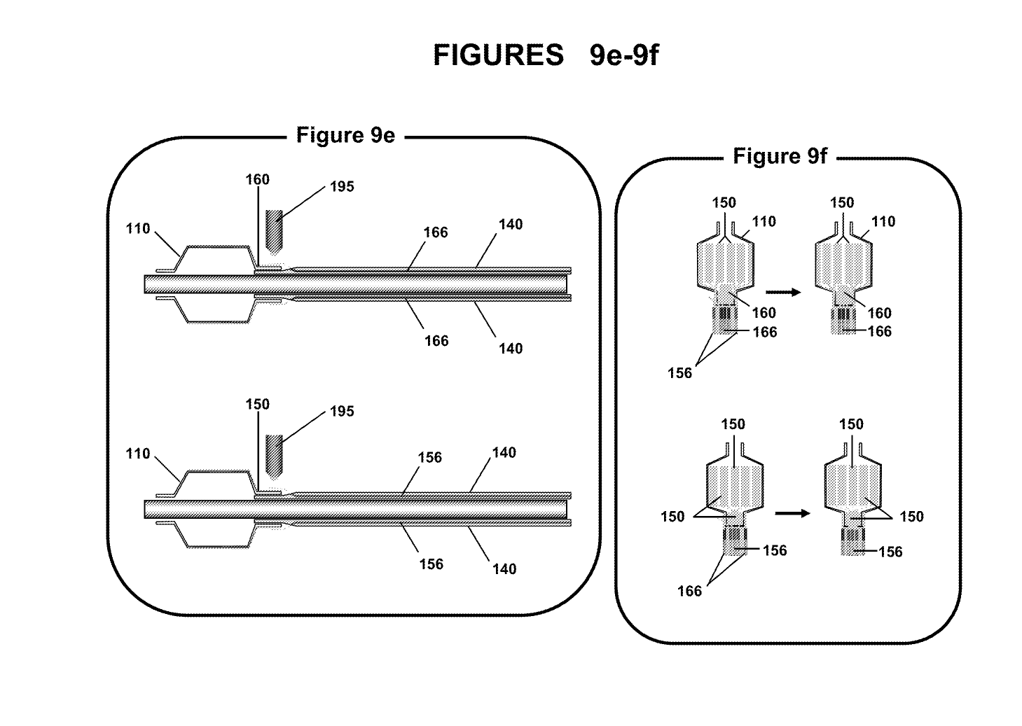

In a second arrangement, shown in FIGS. 7-8, the proximal end of the balloon 110 is inserted into the distal end of the outer shaft 140. This arrangement creates a greater profile at the distal end of the catheter, but makes it easier to solder the electrical leads to their respective electrodes (see description below of FIGS. 9a-9f). FIGS. 7a-7b are cross-sectional side views of the distal end of a balloon catheter 100 showing an example of how the electrical leads 156 and 166 can be embedded in the outer shaft 140. FIG. 7a shows the electrical leads 166 for the counter electrodes 160, while FIG. 7b shows the electrical leads 156 for the gold electrodes 150.

FIGS. 8a-8b are cross-sectional side views of the distal end of a balloon catheter 100 showing an example of how the electrical leads 156 and 166 can be winded around the inner shaft 130 in a helical formation. FIG. 8a shows the electrical leads 156 connect to the gold electrodes 150 on the balloon 110, while FIG. 8b shows the electrical leads 166 connecting to a counter electrode 160. In either example of this second arrangement, the electrical leads 156 and 166 are manageable from the proximal side of the catheter.

FIGS. 9a-9d how an example of how the electrical leads 156 and 166 are connected to the gold electrodes 150 and counter electrodes 160, respectively, using fusion bonding and a solder. In this example, the first electrode design of the balloon catheter embodiment is used (see FIGS. 1a-1d). FIGS. 9a-9b show a balloon catheter 100 according to the first arrangement in which the distal end of the outer shaft 140 is inserted into the proximal end of the balloon 110. FIGS. 9c-9d show a balloon catheter 100 according to the second arrangement in which the proximal end of the balloon 110 is inserted into the distal end of the outer shaft 140.

FIGS. 9e-9f show an example of how the electrical leads 156 and 166 are connected to the gold electrodes 150 and counter electrodes 160 after they have been fusion bonded by using a gold dispenser 195. In this example, the first electrode design of the balloon catheter embodiment is used (see FIGS. 1a-1d) according to the first arrangement in which the distal end of the outer shaft 140 is inserted into the proximal end of the balloon 110.

FIG. 9a shows two cross-sectional side views of the distal end of the balloon catheter 100. These side views of the balloon catheter 100 are taken from perpendicular angles. In the first side view (1), an insulated portion of the counter electrode leads 166 are shown embedded in the outer shaft 140. At a point near to where the distal end of the outer shaft 140 is inserted into the proximal end of the balloon 110, the electrical leads 166 exit through the top of the outer shaft 140 and are fusion bonded to the counter electrodes 160. A thin solder film 190 is used in the fusion bonding process, as well as heat shrink tubing 170 to help heat and melt the thin solder film 190. In the second side view (2), the same fusion bonding process is shown for the gold electrode leads 156 and the gold electrodes 150. FIG. 9b shows two separate series of top views of the distal end of the catheter 100 demonstrating the fusion bonding and soldering processes taking place in FIG. 9a. The first top view (1) shows these processes for the counter electrode leads 166 and a counter electrode 160, while the second top view (2) shows these processes for the gold electrode leads 156 and the gold electrodes 150.

The balloon catheter embodiment shown in FIGS. 9c-9d is according to the second arrangement in which the proximal end of the balloon 110 is inserted into the distal end of the outer shaft 140. The benefit of this second arrangement is that the it makes bonding the electrical leads 156 and 166 to the gold electrodes 150 and counter electrodes 160 easier. This is partially because it is easier to remove the electrical leads 156 and 166 from the outer shaft 140. FIG. 9c shows two cross-sectional side views of the distal end of the balloon catheter 100. These side views of the balloon catheter 100 are taken from perpendicular angles. The first side view (1) shows the counter electrode leads 166 connecting to the counter electrodes 160, while the second side view (2) shows the gold electrode leads 156 connecting to the gold electrodes 150. FIG. 9d shows two separate series of top views of the distal end of the balloon catheter 100 demonstrating the fusion bonding and soldering processes taking place in FIG. 9c. The first top view (1) shows these processes for the counter electrode leads 166 and a counter electrode 160, while the second top view (2) shows these processes for the gold electrode leads 156 and the gold electrodes 150.

In FIGS. 9e-9f, the balloon catheter 100 shows the electrical leads 166 and 156 after they have been fusion bonded to the gold electrodes 150 and counter electrodes 160. No solder film is used. Instead, a gold dispenser 195 dispenses gold to help connect the electrical leads to their respective electrodes. FIG. 9e shows two cross-sectional side views of the distal end of the balloon catheter 100. These side views of the balloon catheter 100 are taken from perpendicular angles. The first side view (1) shows the counter electrode leads 166 connecting to the counter electrodes 160 from gold dispensed from the gold dispenser 195, while the second side view (2) shows the gold electrode leads 156 connecting to the gold electrodes 150 in a similar manner. FIG. 9f shows two separate series of top views of the distal end of the catheter 100 demonstrating the gold dispensing process taking place in FIG. 9e. The first top view (1) shows the gold dispensing process connecting the counter electrode leads 166 to a counter electrode 160, while the second top view (2) shows the gold dispensing process connecting the gold electrode leads 156 to the gold electrodes 150.

In an embodiment of the invention, a stent delivery system 200 is used in conjunction with the drug delivery system described above. The stent delivery system comprises self-expandable stent scaffolding 210, gold electrodes 250 loaded onto the stent scaffolding 210, counter electrodes 260, a sheath 220, an outer shaft 240, and an inner shaft 230 with a guidewire lumen 235, a guidewire 236, and a radiopaque marker band 265.

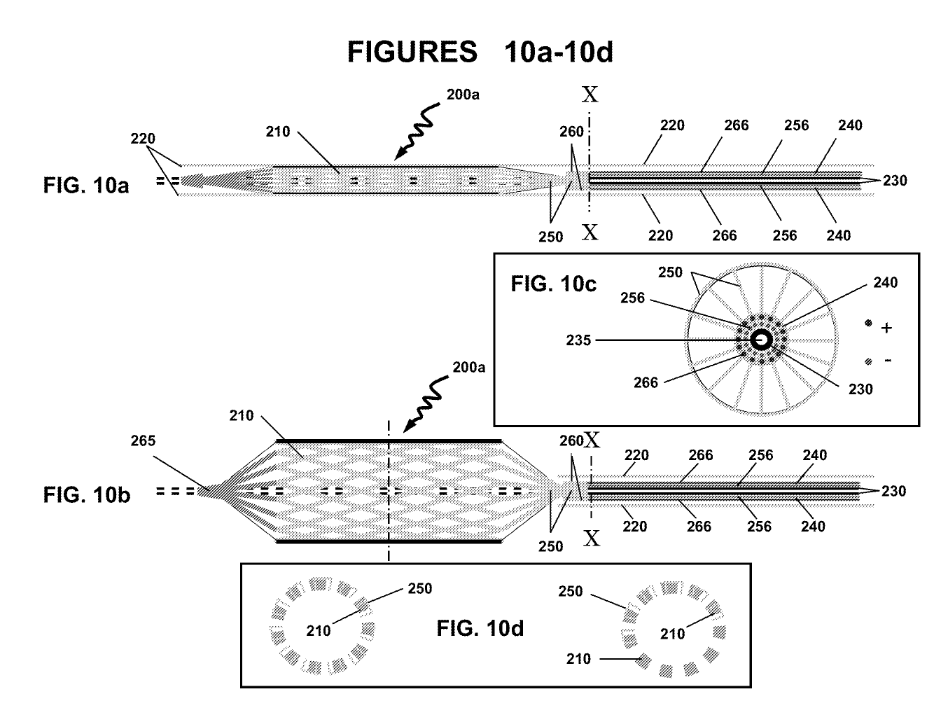

Various stent delivery system designs may be used with the drug delivery system, though four are described. In all of the following designs in FIGS. 10-13, the gold electrodes 250 comprise electrically conductive plates comprising gold or layered with a thin gold film which are circumferentially located on the outside of the scaffolding 210. Current travels from a current source near the proximal end of the system through the counter electrode leads 266 to counter electrodes 260. Some portion of the counter electrodes 260 are located on the exterior of the distal end of the outer shaft 240 to ensure the counter electrodes 260 can easily contact the surrounding blood or body fluid. Though in the electrode designs shown below, two counter electrodes 260 are shown on opposite sides of the exterior of the distal end of the outer shaft 240, it should be understood that more counter electrodes 260 (or even a single counter electrode 260) may be used. The counter electrodes 260 are positively charged with regard to the gold electrodes 250. The current travels from the counter electrodes 260 through the blood or body fluid to the gold electrodes 250 located on the scaffolding 210. To prevent the current from traveling into the human body, the counter electrodes 260 should be located as close to the some portion of the gold electrodes 250 as possible, without actually contacting the counter electrodes 260 and gold electrodes 250 together. Preferably, the distance between the portions of the counter electrodes 260 and gold electrodes 250 nearest each other is between 0.01 mm-100 mm. The gold electrodes 250 are loaded with the biologically active materials described above. The radiopaque marker band 265 is shown situated external to the distal end of the balloon 210. The electrical leads 256 and 266 should be covered with insulation as much as possible to prevent electricity leakages.

In FIGS. 10a-10d, a self-expandable stent scaffold 210 with gold electrodes 250 is used in an over-the-wire type stent delivery system 200a. The gold electrodes 250 are located on the distal end of the outer shaft 240 and on the outside of the scaffolding 210. The gold electrodes 250 may be either circumferentially loaded along the outside of the scaffolding 210, or may be only partially loaded (see FIG. 10d). The counter electrodes 260 are located at the distal end of the outer shaft 240 and are separated from the gold electrode 250 portions at the distal end of the outer shaft 240 by an insulating material. FIG. 10a is a side perspective view of the distal end of the delivery system 200a when the scaffolding 210 is not expanded. The scaffolding 210 is folded inside a sheath 220 during delivery to the treatment site. The portion of this view to the right of the dotted line X-X is a cross-sectional view of the outer and inner shafts showing electrical leads 256 and 266 traveling the length of the stent delivery system 200a. Electrical leads 256 connect to the gold electrode 250, and the electrical leads 266 connect to the counter electrodes 260. FIG. 10b shows the stent delivery system 200a from a side perspective view when the scaffolding 210 is expanded. The scaffolding 210 covered with gold electrodes 250 self-expands once the sheath 220 is pulled back. Electrical leads 266 leading from the counter electrodes 260 are embedded in the outer shaft 240, while electrical leads 256 from the gold electrodes 250 travel in the area between the outer shaft 240 and inner shaft 230. Thus, the electrical leads 266 and 256 from the counter electrodes 260 and gold electrodes 250 run parallel to each other. FIG. 10c shows a cross-sectional view of the stent delivery system 200a along the dotted line X-X in FIGS. 10a and 10b. The gold electrodes 250 continuously cover the outside of the stent scaffolding 210. The counter electrodes 260 cannot be seen. Electrical leads 256 connect to the gold electrodes 250 at the proximal end of the stent scaffolding 210 and are embedded in the area between the outer shaft 240 and inner shaft 230. Electrical leads 266 from the counter electrodes 260 are embedded in the outer shaft 240. A guidewire lumen 235 is shown, though a guidewire is not. FIG. 10d is a cross-sectional view of the scaffolding 210. The stent scaffolding 210 is shown circumferentially loaded or alternatively partially loaded with gold electrodes 250.

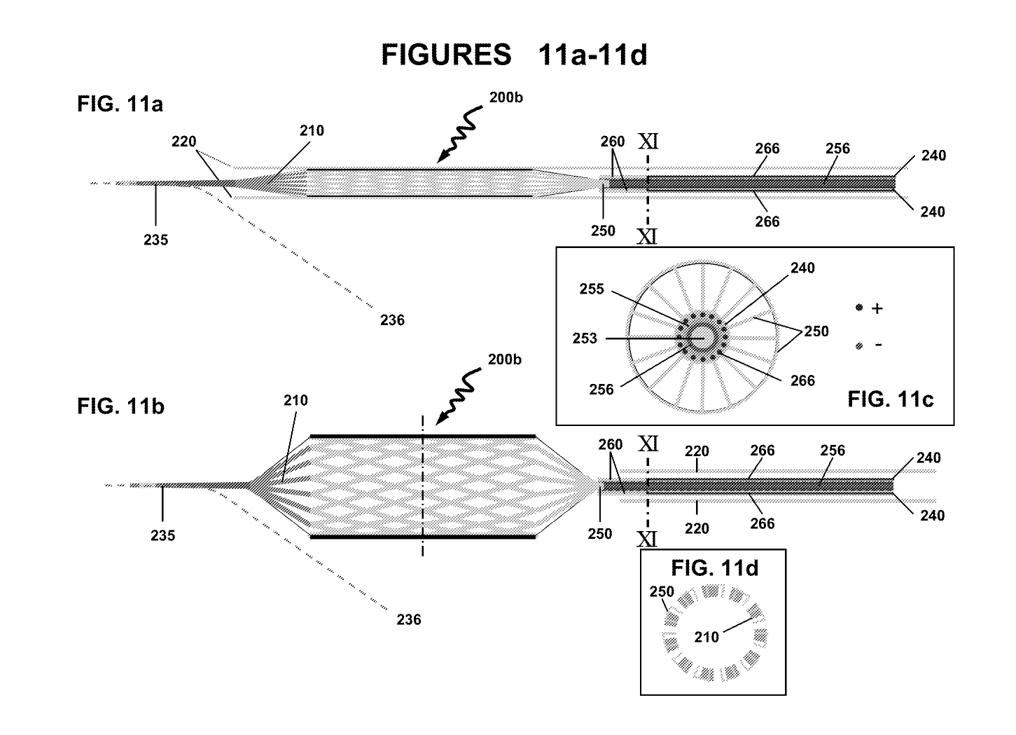

In FIGS. 11a-11d, a self-expandable stent scaffold 210 with electrodes 250 is used in a rapid exchange type stent delivery system 200b. The gold electrodes 250 are located on the distal end of the outer shaft 240 and on the outside of the scaffolding 210. The gold electrodes 250 are shown circumferentially loaded along the outside of the scaffolding 210 (see FIG. 11d). FIG. 11a shows the stent delivery system 200b from a side perspective view when the scaffolding 210 is not expanded and is still inside the sheath 220. The scaffolding 210 is folded inside a sheath 220 during delivery to the treatment site. The sheath portion of this view to the right of the dotted line XI-XI is a cross-sectional view of the outer and inner shafts 240 and 230 showing the electrical leads 256 and 266 traveling the length of the stent delivery system 200b. The guidewire 236 extends beyond the scaffolding 210. FIG. 11b shows the stent delivery system 200b from a side perspective view when the scaffolding 210 is expanded. The sheath portion of this view to the right of the dotted line XI-XI is a cross-sectional view of the outer and inner shafts 240 and 230 showing the electrical leads 266 and 256 traveling the length of the stent delivery system 200b. The scaffold 210 covered with gold electrodes 250 self-expands once the sheath is pulled back. FIG. 11c shows a vertical cross-sectional view of the stent delivery system 200b along the dotted line XI-XI in FIGS. 11a and 11b. The gold electrodes 250 continuously cover the outside of the stent scaffolding 210. The counter electrodes 260 cannot be seen. Counter electrode leads 266 are embedded in the outer shaft. A layer of insulation 255 is used in the area between the outer shaft 240 and inner shaft 260. Gold electrode leads 256 are embedded in the inner shaft 230. A core wire 253 travels in the area inside the inner shaft. FIG. 11d shows a cross-sectional view of the scaffolding 210. The stent scaffolding 210 is shown circumferentially loaded.

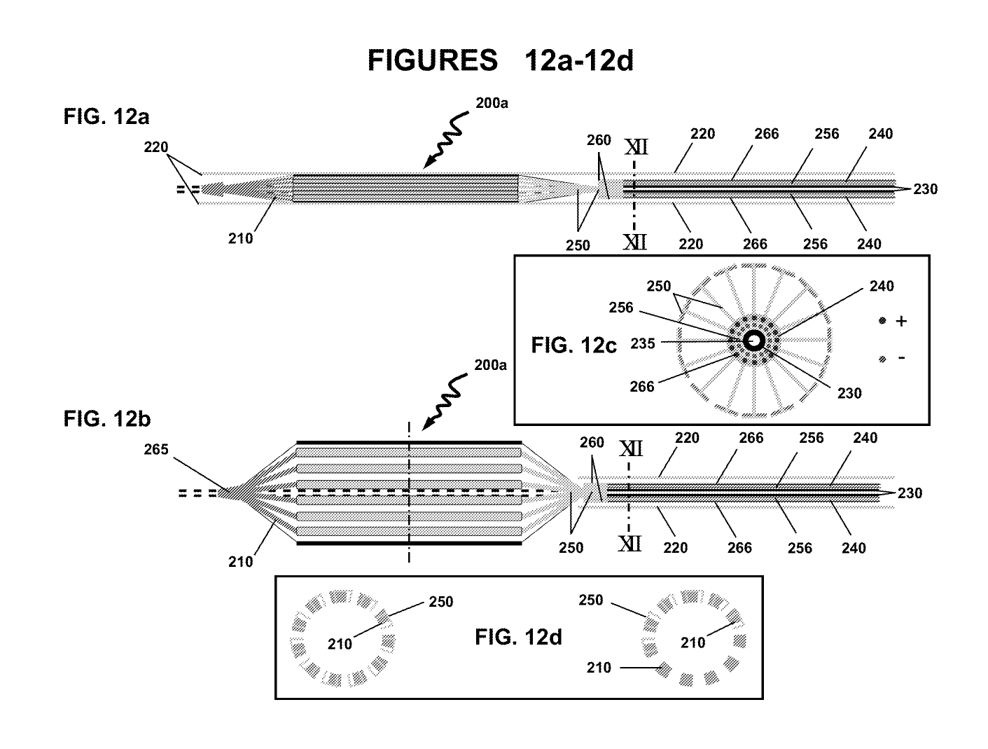

FIGS. 12a-12d shows an over-the-wire type stent delivery system 200a similar to the ones in FIGS. 10a-10d. The difference is that the stent scaffolding design is not a cross-mesh design, but instead utilizes parallel rectangular bars which do not interlock over the length of the scaffold 210 except at the proximal and distal ends of the scaffold 210. FIG. 12a shows the stent delivery system 200a from a side perspective view when the scaffolding 210 is not expanded. The sheath portion of this view to the right of the dotted line XII-XII is a cross-sectional view of the outer and inner shafts 240 and 230 showing the electrical leads 256 and 266 traveling the length of the stent delivery system 200a. FIG. 12b shows the stent delivery system 200a from a side perspective view when the scaffolding 210 is expanded. The sheath portion of this view to the right of the dotted line XII-XII is a cross-sectional view of the outer and inner shafts 240 and 230 showing the electrical leads 256 and 266 traveling the length of the stent delivery system 200a. FIG. 12c shows a cross-sectional view of the stent delivery system along the dotted line XII-XII in FIGS. 12a and 12b. FIG. 12d shows a cross-sectional view of the scaffolding 210. The stent scaffolding 210 is shown circumferentially loaded or alternatively partially loaded with gold electrodes 250.

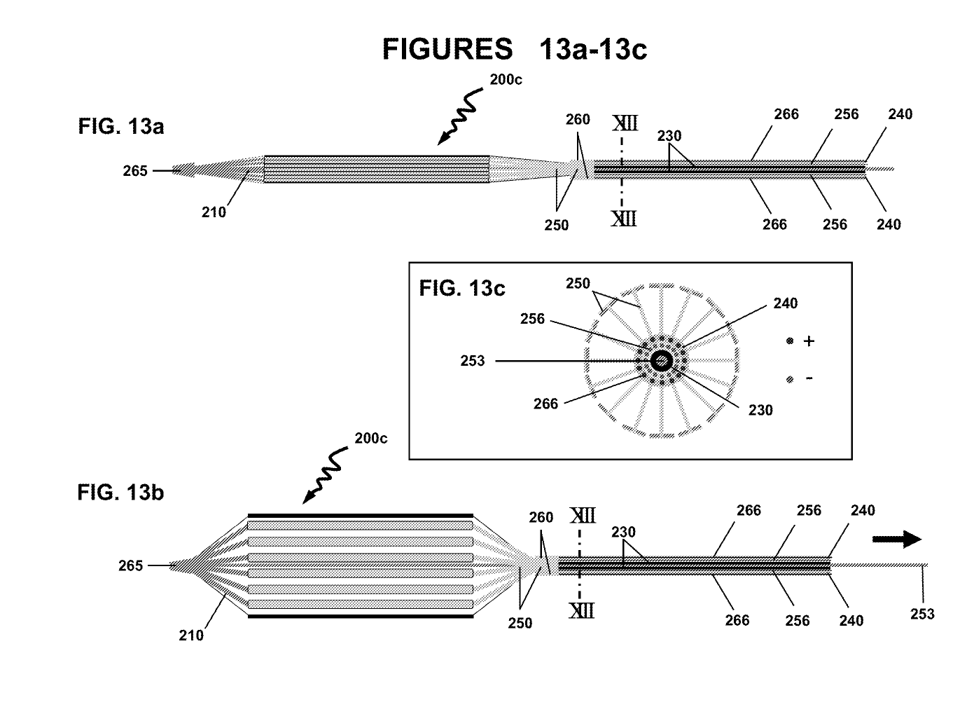

FIGS. 13a-13b show side perspective views of the distal end of a stent scaffold delivery system 200c having an expandable scaffold and electrodes. A core wire 253 is fixed to the radioplaque marker band 265 portion. The stent scaffolding 210 is shown circumferentially loaded or alternatively partially loaded with gold electrodes 250. The gold electrodes 250 are loaded with the bioactive agent described above. Gold electrode leads 256 are attached to the gold electrodes 250 and run from the proximal end of the stent scaffold delivery system 200c to the distal end of the stent scaffold delivery system 200c inside the outer shaft. A counter electrode 260 is situated near the proximal end of the stent scaffold delivery system 200c and has counter electrode leads (not shown) running from the distal end of the stent scaffold delivery system 200c to the proximal end of the stent scaffold delivery system 200c. FIG. 13a shows the stent scaffold delivery system during delivery when the core wire 253 is not withdrawn, while FIG. 13b shows the stent scaffold delivery system after the core wire 253 has been withdrawn. FIG. 13c shows a cross-sectional view of the stent scaffold delivery system along the dotted line XIII-XIII in FIGS. 13a and 13b.

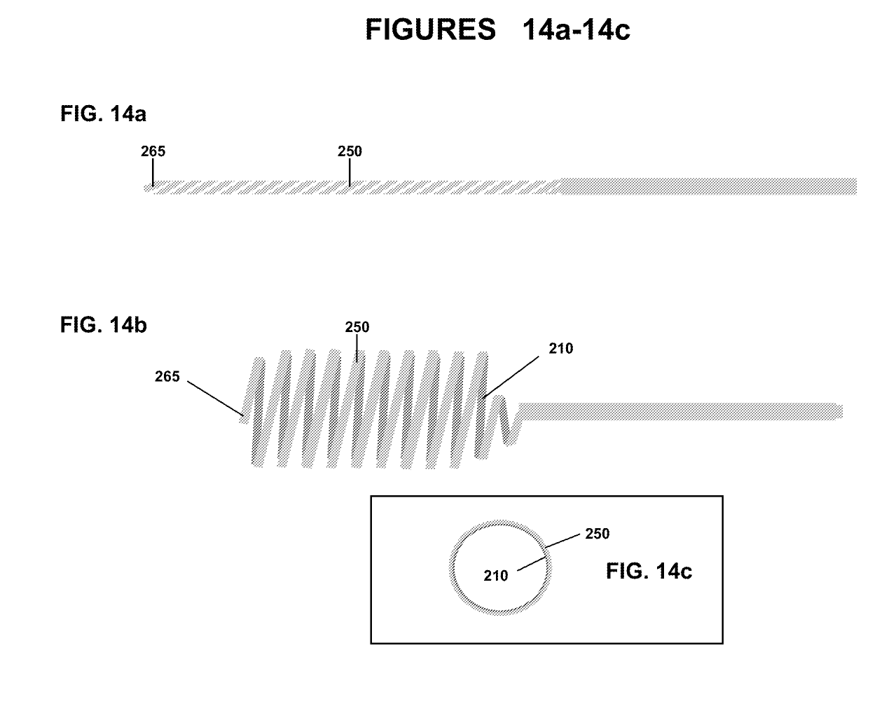

FIGS. 14a-14c show a shape memory scaffold 210 that can be used in the examples above instead of a self-expandable scaffold. FIGS. 14a-14b are side perspective views of the scaffold. As shown in FIG. 14a, the shape memory scaffolding 210 is a compact, narrow coil during delivery. The gold electrodes 250 are circumferentially located on the outside of the shape memory scaffolding 210. In FIG. 14b, the shape memory scaffolding is expanded by an inflated balloon (not shown) into a larger coil shape. A radioplaque marker band 265 is situated at the end of the shape memory scaffold 210. FIG. 14c approximately shows a cross-sectional view of the shape memory scaffold 210. The scaffolding 210 is shown loaded continuously with gold electrodes 250.

### General Methods and Procedures:

The device of the present application may be made in a number of steps, including the preparation or synthesis of biodegradable polymers with the reactive end group; the preparation of the nanoparticle comprising the biologically active agent or drug along with the biodegradable polymer. The substrate comprising the polymer that comprises a nanoparticle, microgranulated particle or microsphere may then be immobilized on the device.

### Amine-Terminated Biodegradable Polymers:

The biodegradable polymers with amino groups (i.e., amine-terminated biodegradable polymers) may be prepared starting with a number of different commercially available polymers with carboxylic acid groups. Such polymers may include PCL, PAE, PLLA, PLA, PLGA-COOH. The carboxylic acid may be condensed with an amine, such as NH₂—(CH₂CH₂O)ₙ—NH₂ that is commercially available. For example, PLGA-COOH (10 g, 0.11 mmol) in DCM (50 mL) was treated with DCC (45.4 mg, 0.22 mmol) and NHS (25.3 mg, 0.22 mmol) at room temperature for about 12 hours, and the resulting activated PLGA product (PLGA succinamidyl derivative) was filtered and then precipitated our with anhydrous diethyl ether. The resulting activated PLGA, as a solid is dried under vacuum.

In the second step, activated PLGA (10 gm), hexamethyleneglycol-diamine (750 mg) and DMSO (anhydrous, 100 ml) was combined and stirred at room temperature for about 12 hours. The resulting solid was filtered. The solution was added dropwise into a solution of cold ethanol, and the precipitation was filtered and washed with cold ethanol (3×1 L), and then dried under vacuum to form PLGA-C(O)NH—(CH₂CH₂O)ₙ—NH₂. The hydrophobic fragment of the substrate prevents the nanoparticles (microspheres, or microgranulated particles) from re-adsorption onto the substrates when they are release, that allows the particles to penetrate into the tissues.

The biodegradable polymers may also be based on different homopolypeptides having an amine group for condensation or coupling reaction, such as arginine, lysine and histidine.

The biodegradable polymer be functionalized or may terminate in a compound, such as biotin. The preparation for such compounds is based on the reaction of a PEG amino-alcohol, such as commercially available HO—(CH₂CH₂O)ₙ—NH₂ with NHS-biotin (also commercially available) to form the corresponding HO—(CH₂CH₂O)ₙ-biotin coupled product. A subsequent reaction of the biotin coupled product with the biodegradable polymer, such as PLGA-COOH in a solvent, such as refluxing toluene, provide the PLGA coupled product, PLGA-COO—(CH₂CH₂O)ₙ-biotin.

Similarly, the corresponding reactions as described above, to form a biodegradable polymer such as PLGA-COO—(CH₂CH₂O)ₙ-avidin, provides the coupled product with high specificity and high affinity, and as further described below.

### Preparation of Biodegradable Nanoparticles:

Using the above described processes, shell compositions such as PLGA-CONH—(CH₂CH₂O)ₙ—NH₂, as represented below, may form biodegradable nanoparticles with biologically active agents, such as antithrombotics, antiproliferatives, anti-inflammatory agents, smooth muscle cell migration inhibitors and restenosis-reducing agents. Such agents may include, for example, paclitaxel, sirolimus and simvastatin.

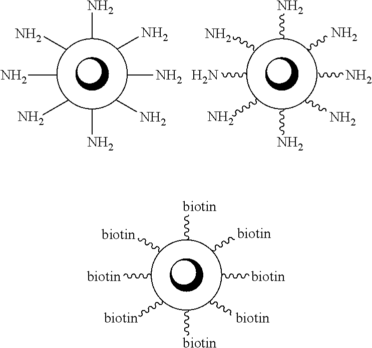

Nanoparticle-1 (NP-6-1) Nanoparticle-2 (MP-3000-1) Nanoparticle-2 (NP-3000-bio)

### Procedure for the Preparation of the Nanoparticles:

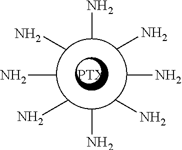

### Example

### NP-6-1 Preparation Conjugated with Paclitaxel (PTX)

An organic solution of PLGA (100 mg) and paclitaxel (0.4 or 1 mg) in acetone (10 ml) was added to an aqueous poloxamer 188 solution (10 or 20 ml, 0.25% w/v) under magnetic stirring at room temperature. Following 15 min of magnetic stirring the acetone was removed under reduced pressure. To remove the non-incorporated drug, the obtained nanosuspension was filtered (S&S ‘Filter paper circles’, pore size 1 μm) and ultra-centrifuged twice at 61 700×g for 1 h at 4° C. (Beckman L-80 ultracentrifuge equipped with a Ti-70 rotor). The supernatant containing the free drug was discarded and the pellet was freeze-dried for 24 h (Labconco Freeze Dry System—Freezone 6 Liter. Kansas City, Mo. USA). *W/a surfactant, Pluronic.

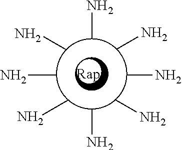

NP-3000-1 Preparation Conjugated with Rapamycin (“Rapa”):

Nanoparticles were prepared using the salting-out method in which acetone was chosen as the water-miscible organic solvent, because of its pharmaceutical acceptance with regard to toxicity. Typically, an acetone solution (3.5 g) containing 3 wt. % PEO-PLGA and various amounts (0-1.2 wt %) of drug was emulsified under mechanical stirring (20,500 rpm; 40 s: T25 Ultraturrax equipped with an S25 dispersing tool, Ika-Labortechnik, Staufen, Germany) in an aqueous phase (8.75 g) containing 60 wt. % MgCl₂.6H₂O as the salting-out agent (in a glass beaker 3.5 cm diameter; 6.6 cm height). After the fast addition (5 s) of pure water (7.5 g) under mechanical stirring (20,500 rpm) causing acetone to diffuse into the water phase, nanoparticles were formed and stirring was continued (20,500 rpm; 20 s). The nanoparticles were purified by rinsing with water. First, the nanoparticles were separated by ultracentrifugation (65,000×g for 30 min; Centrikon T-2180, Kontron Instruments, Watford, UK) and the supernatant was removed. The nanoparticles were redispersed in water, centrifuged and the supernatant was removed. This procedure was repeated three times. *w/o using a surfactant.

### Example

### NP-3000-Bio Preparation Conjugated with Rapamycin

Nanoparticles are produced using a single emulsion technique in which 10 mL of a 25-mg/mL solution of the polymer and various amounts of drug in dichloromethane is homogenized for 2 min in 250 mL of a 0.1% aqueous PVA solution (PVA 88% hydrolyzed, PolyScience Inc., Warrington, Pa.). The resulting emulsion is stirred for 4 h to allow the dichloromethane to evaporate. The nanoparticles are collected by centrifugation at 5,000 rpm for 10 min and washed three times in distilled water and then lyophilized.

### Immobilization on the Gold (Au) Surface Layer:

The device upon which an ultrathin gold film has been formed or deposited upon, is submerged for 18 hours in a 1-mM ethanol solution of HOOC-PEG-C₅₋₁₈alkylenyl-SH (or also 11-carboxyl-1-undecanethiol), that induces the formation of a self-assembled monolayer (SAM) on the gold surface. A gold-sulfur bond (Au—S) is formed between the thiol group (—SH) and the gold surface, wherein the tail of the SAM terminates with a carboxyl or carboxylic acid group. The terminal carboxyl group is induced to react for 2 hours at room temperature with 0.2 M EDC/0.5 M N-hydroxy succinimide, so that the carboxyl group is succinimidated or forms a succinimidyl derivative. This succinimidyl derivative is also allowed to react for 2 hours at room temperature with nanoparticles comprising paclitaxel or rapamycin (see representation below), and the paclitaxel (or rapamycin) containing biodegradable nanoparticles are bonded to the Au substrate surface by a covalent bond. The nanoparticles may be a poly(lactic/glycolic) acid copolymer (PLGA) terminating with an amino group, as shown below.

Similarly, the device upon which an ultrathin gold film has been formed or deposited upon, is submerged for 18 hours in a 1-mM ethanol solution of HOOC-PEG-C₅₋₁₈alkylenyl-SH (or also 11-carboxyl-1-undecanethiol), that induces the formation of a self-assembled monolayer (SAM) on the gold surface. A gold-sulfur bond (Au—S) is formed between the thiol group (—SH) and the gold surface, wherein the tail of the SAM terminates with a carboxyl or carboxylic acid group. The terminal carboxyl group is induced to react for 2 hours at room temperature with 0.2 M EDC/0.5 M N-hydroxy succinimide, so that the carboxyl group is succinimidated or forms a succinimidyl derivative. This succinimidyl derivative is also allowed to react for 2 hours at room temperature with avidin to form the immobilized avidin substrate on the gold surface. The nanoparticles encapsulating or comprising rapamycin bonded to biotin, prepared according to the method noted above, may be added to the avidin immobilized on the substrate that is bonded to the gold surface layer, and the biotin complexes with avidin through their well known strong affinity for complexation by a molecular biorecognition phenomenon, as shown below.

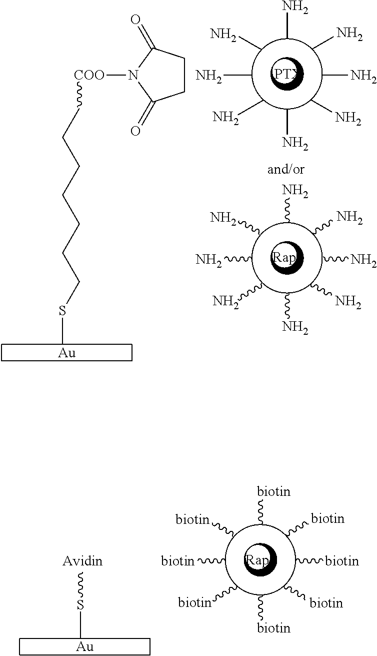

### Experimental Methods:

Manufacturing a balloon-type device for local intraluminal transport of a biologically and physiologically active agent:

An ultrathin gold film, that may be used as an electrode lead wire, is formed or coated on a percutaneous transluminal coronary angioplasty (PTCA) balloon, either by applying gold to the entire surface of the balloon, or a part of the surface of the balloon, such as the exterior surface, uniformly by an ultrafine ink jet technique or in a predetermined pattern, while the balloon is maintained in a dilated state. The gold electrode may also be fabricated using optical lithography as is known in the art. The balloon, whereupon an ultra thin gold film has been formed, is submerged for 18 hours in a 1-mM ethanol solution of 11-carboxyl-1-undecanethiol having a hydrophobic alkane chain inducing formation of a self-assembled monolayer (SAM) terminating in a carboxyl group, wherein the thiol end of the compound forms a sulfur-gold bond (S—Au) on the gold-coated layer. In one aspect, in order to ensure the delivery of an effective amount of the biologically active agent, the area for the formation of the gold surface layer is at least about 20% of the surface area of the device, such as the balloon or scaffold that will be in contact with the intraluminal surface when the balloon or scaffold is expanded or deployed, and the substrate comprising the biologically active agent is delivered at the desired location. In certain variations, the formation of the gold surface layer is at least about 25%, at least 30%, at least 40%, at least 50%, at least 75%, at least 90% or at least 95% or more of the surface area of the balloon (or scaffold) that will be in contact with the intraluminal surface. Additionally, the area for the formation of the SAM on the gold surface layer is at least about 20% of the surface area of the gold surface layer that will be in contact with the intraluminal surface when the balloon (or scaffold) is expanded or deployed, and the substrate comprising the biologically active agent is delivered at the desired location. In certain variations, the formation of the SAM on the gold surface layer is at least about 25%, at least 30%, at least 40%, at least 50%, at least 75%, at least 90% or at least 95% of the surface area of the gold surface layer that will be in contact with the intraluminal surface.

Next, the terminal carboxyl group is allowed to react for 2 hours with 0.2 M EDC/0.5 M N-hydroxy succinimide, so that the carboxyl group is succinimidated or forms the succinimidyl derivative. This succinimidated derivative is also allowed to react for 2 hours with granules comprising sirolimus capable of inhibiting smooth-muscle proliferation, and these sirolimus-containing biodegradable granules are attached to the balloon surface by covalent bonding.

In a similar procedure as described above, an ethanol solution of a carboxy-PEG-C₅₋₁₈alkyl-thiol or a carboxy-PEG-thiol may be used in place of the 11-carboxyl-1-undecanethiol to form the corresponding carboxyl terminated compound that may derivatized to the corresponding succinimidyl derivative for a subsequent coupling reaction as provided above.

Also, in a similar procedure as described above, a poly(lactic/glycolic) acid copolymer (PLGA) comprising a biologically active agent, and terminating in an amino group is employed to couple with the above succinimidyl derivative to form the corresponding amides. These PLGA derivatives form microparticles, microgranulated particles or microspheres that encapsulate the biologically active agents.

Manufacturing a device for local intraluminal transport of a biologically and physiologically active agent using a shape memory alloy:

An ultrathin gold film, acting as an electrode lead wire, is formed on part of a coronary scaffold by heating the part of the coronary scaffold comprising a shape memory alloy and applying gold to the entire surface of the part of the coronary scaffold while it is maintained in a dilated state, uniformly by an ultrafine ink jet technique or in a predetermined pattern. This part of the coronary scaffold, whereupon an ultra thin gold film has been formed, is submerged for 18 hours in a 1-mM ethanol solution of 11-carboxyl-1-undecanethiol having a hydrophobic alkane chain inducing the formation of a self-assembled monolayer (SAM) terminating in a carboxyl group, wherein the thiol end of the compound forms a sulfur-gold bond (S—Au) on the gold-coated layer. Next, the terminal carboxyl group is allowed to react for 2 hours with 0.2 M EDC/0.5 M N-hydroxy succinimide, so that the carboxyl group is succinimidated or forms the succinimidyl derivative. This succinimidated derivative is also allowed to react for 2 hours with granules comprising sirolimus capable of inhibiting smooth-muscle proliferation, and these sirolimus-containing biodegradable granules are attached to the exterior surface of the coronary scaffold by covalent bonding.

In a similar procedure as described above, an ethanol solution of a carboxy-PEG-C₅₋₁₈alkyl-thiol or a carboxy-PEG-thiol may be used in place of the 11-carboxyl-1-undecanethiol to form the corresponding carboxyl terminated compound that may derivatized to the corresponding succinimidyl derivative for a subsequent coupling reaction as provided above.

Also, in a similar procedure as described above, a poly(lactic/glycolic) acid copolymer (PLGA) comprising a biologically active agent, and terminating in an amino group is employed to couple with the above succinimidyl derivative to form the corresponding amides. These PLGA derivatives form microparticles, microgranulated particles or microspheres that encapsulate the biologically active agents.

In particular variations of the methods as provided herein, at lest part of the counter electrode that is on the surface of the balloon or scaffold does not directly come into contact with the intraluminal surface when the balloon or scaffold is deployed.

Manufacturing a self-dilating, retractable device for local intraluminal transport of a biologically and physiologically active agent using a superelastic alloy:

The mesh-patterned part and the entire inner surface part of a coronary scaffold comprising a shape memory alloy are masked. An ultrathin gold film, acting as an electrode lead wire, is formed on this mesh-patterned part of the coronary scaffold by heating it, after it has undergone the aforementioned process, and applying gold uniformly by chemical vapor deposition to the entire surface part of the coronary scaffold while it is maintained in a dilated state. This part of the coronary scaffold, where upon an ultrathin gold film has been formed, is submerged for 18 hours in a 1-mM ethanol solution of 11-carboxyl-1-undecanethiol having a hydrophobic alkane chain (e.g., a C₅₋₁₈alkylenyl group) inducing the formation of a self-assembled monolayer (SAM) terminating in a carboxyl group, wherein the thiol end of the compound forms a sulfur-gold bond (S—Au) on the gold-coated layer. Next, the terminal carboxyl group is allowed to react for 2 hours with 0.2 M EDC/0.5 M N-hydroxy succinimide, so that the carboxyl group is succinimidated or forms the succinimidyl derivative. This succinimidated derivative is also allowed to react for 2 hours with granules comprising simvastatin, which stabilizes vulnerable plaque, and polyarginine having an HIV-TAT sequence, and these simvastatin-containing biodegradable granules are attached to the exterior surface part of the coronary scaffold by covalent bonding.

In a similar procedure as described above, an ethanol solution of a carboxy-PEG-C₅₋₁₈alkyl-thiol or a carboxy-PEG-thiol may be used in place of the 11-carboxyl-1-undecanethiol to form the corresponding carboxyl terminated compound that may derivatized to the corresponding succinimidyl derivative for a subsequent coupling reaction as provided above.

Also, in a similar procedure as described above, a poly(lactic/glycolic) acid copolymer (PLGA), comprising a biologically active agent, and terminating in an amino group is employed to couple with the above succinimidyl derivative to form the corresponding amides. The PLGA derivatives form microparticles or microgranulated particles that encapsulate the biologically active agents.

### Surface Modification of Nanoparticles:

### Formulation of Nanoparticles:

Nanoparticles are formulated by an oil-in-water emulsion solvent evaporation technique as described elsewhere. In brief, PLGA (200 mg) and a biologically active agent (40 mg) are co-dissolved in 10 mL of methylene chloride. The organic phase is emulsified in an aqueous poly(vinyl alcohol) solution (2% w/w, 40 mL, adjusted to pH 8.0 with sodium phosphate dibasic) using sonication (10 min, 55 W, SONICATOR (model XL2020, Misonic Inc., Farmingdale, N.Y.) to form an oil water emulsion. The emulsion is stirred overnight to evaporate organic solvent. Nanoparticles thus formed are recovered by ultracentrifugation at 140000 g using a Beckman Ultracentrifuge (model LE 80, Schaumburg, Ill.), are washed 3 times with water to remove PVA and the unencapsulated biologically active agent, and is lyophilized for 48 h. Nanoparticles with higher biologically active agent loading are formulated by using an appropriate amount of the active agent, as calculated from the encapsulation efficiency.

### Surface Modification of Nanoparticles:

Three different methods for nanoparticle surface modification are described below:

Chemical Coupling: The procedure involved two steps, activation of the preformulated nanoparticles with an epoxy compound followed by the reaction with surface modifying agents.

(A) Surface Activation: A sample of the nanoparticles (40 mg) as prepared according to the method described herein, is suspended in 5 mL of borate buffer (50 mM, pH 5) by sonication at 55 W for 30 s over an ice bath. Zinc tetrafluoroborate hydrate (12 mg, as a catalyst), is added to the nanoparticle suspension, followed by the addition of a Denacol solution (14 mg in 2 mL of borate buffer). After 30 min of reaction at 37° C. with stirring, the nanoparticles are separated by ultracentrifugation and washed 3 times with water to remove unreacted Denacol. The epoxy activated nanoparticles are coupled to surface modifying agents as described below.

(B) Coupling Reaction: Nanoparticles surface activated as above (40 mg) are suspended in 20 mL of borate buffer. A solution of heparin (14 mg, activity 160 units/mg) in 4 mL of borate buffer is added to the nanoparticle suspension with stirring at 37° C. The reaction is carried out for 2 h with low speed stirring. For quantitation purposes, 3H-labeled heparin is used. The unreacted heparin was removed by ultracentrifugation followed by extensive dialysis against water over 26 h or until there is no further leaching of heparin. Nanoparticles are lyophilized for 48 h.

Co-incorporation of Surface Modifying Agents into Nanoparticles:

In this procedure, surface modifying agents are co-incorporated into the nanoparticle matrix during the nanoparticle formulation protocol. For example, to formulate nanoparticles containing isobutyl cyanoacrylate, a surface modifying agent, polymer (PLGA, 108 mg) and the surface modifying agent (36 mg) (PLGA to cyanoacrylate ratio 4:1) are dissolved in 5 mL of methylene chloride. The biologically active agent, as disclosed herein, is dissolved in the above polymer solution and then emulsified into a PVA (25 mL, 2.5%, pH 8.0) solution by sonication as above to form an oil water emulsion. The emulsion is stirred overnight to evaporate the organic solvent, and nanoparticles are recovered by ultracentrifugation as described above. Nanoparticles containing lipid (L-α-phosphatidyl ethanolamine) as a surface modifying agent are also prepared by a similar protocol. The lipid solution in chloroform (4 mg/mL) is mixed with a polymer solution in methylene chloride (20 mg/mL) (lipid-to-polymer ratio was 1:3) and emulsified in a PVA solution as above to form an oil-water emulsion. The nanoparticles are recovered following evaporation of organic solvent as above.

III. Surface Adsorptions: This procedure is used for surface modifying agents which are cationic in nature. Since, the certain unmodified nanoparticles are anionic in nature, mixing of these surface modifying agents with the nanoparticle suspension could result in their ionic bonding to the nanoparticle surface. Surface modifying agents, didodecyldimethylammonium bromide (DMAB) (5%), ferritin (5%), dextran (5%) or lipofectin (2.5%) are dissolved in 10 mL of water to form a solution or colloidal dispersion. Nanoparticles of desired weight are added in each of these solutions so that the required percent of surface modifying agent in relation to weight of the nanoparticles is achieved. For example, to obtain nanoparticles with 5% DMAB, 5 mg of DMAB are dissolved in 10 mL of water and 95 mg of nanoparticles are suspended in the solution containing the surface modifying agent by sonication for 30 s at 55 W of energy output over an ice bath. The suspensions of nanoparticles are then frozen over dry ice and lyophilized for 48 h.

Various agents may be used for surface modification of the nanoparticles are provided in the Table:

TABLE 1
Nanoparticle Surface Modifying Agents

| | Methods of | Reason for |
|---|---|---|
| Agents | modification/Source | modification |
| Heparin (HP) | heparin (HP) chemical | introduce anticoagulant |
| | coupling to nanoparticles | effect |
| | via epoxy activation | |
| L-α-phosphatidylethanolamine (LP) | incorporated into | positively charged lipid |
| | nanoparticles | |
| Cyanoacrylate (CN) | incorporated into | bioadhesive polymer |
| | nanoparticles | |
| Epoxide (EP) | chemical coupling via | cross-linking agent |
| | epoxy reaction to | |
| | partially hydrolyzed | |
| | nanoparticles | |
| Fibronectin (FN) | adsorbed onto | A protein, a natural cell |
| | nanoparticles | adhesive with collagen- |
| | | specific binding |
| Fibrinogen (FG) | adsorbed onto | clotting factor |
| | nanoparticle | |
| Ferritin (FERR) | receptor specific protein | adsorbed onto |
| | | nanoparticle |
| Lipofectin (LP) | positively charged lipid, | adsorbed onto |
| | high cell membrane | nanoparticle |
| | affinity | |
| Didodecyldimethylammonium bromide (DMAB) | cationic detergent | adsorbed onto |
| | | nanoparticle, charge |
| | | affinity |
| DEAE-Dextran (DEAE) | cationic polysaccharide | adsorbed onto |
| | | nanoparticle, charge |
| | | affinity |
| Penetratin^{a} | Protein derived CPP | adsorbed onto |
| RQIKIWFQNRRMKWKK | | nanoparticle |
| Tat^{a} | Protein derived CPP | adsorbed onto |
| CGRKKRRQRRRPPQC | | nanoparticle |
| Pvec^{a} | Protein derived CPP | adsorbed onto |
| LLIILRRRIRKQAHAHSK-amide | | nanoparticle |
| MAP^{a} | Model peptide | adsorbed onto |
| KLALKLALKALKAALKLA-amide | | nanoparticle |
| (Arg)₇^{a} | Model peptide | adsorbed onto |
| RRRRRRR | | nanoparticle |
| MPG^{a} | Designed CPP | adsorbed onto |
| GALFLGFLGAAGSTMGAWSQPKSKRKV | | nanoparticle |
| Transportan^{a} | Designed CPP | adsorbed onto |
| GWTLNSAGYLLGKINLKALAALAKISIL-amide | | nanoparticle |

^{a}Example of cell-penetrating peptides (CPPs) that may be used for surface modification that may be coated or absorbed into the nano-particles. See M. Zorko et al, Advanced Drug Delivery Reviews, 57 (2005), 529-545. The surface modified particles with CPPs are able to penetrate cell membranes and transport the active agents into cells.

The results demonstrated that surface modification of the nanoparticles, when released from the device as prepared according to the present disclosure, improves the arterial levels of the biologically active agents due to enhanced uptake of nanoparticles. The greatest enhancement of uptake was observed with the nanoparticles surface modified with DMAB, DEAE-dextran and Lipofectin. The DMAB surface modified nanoparticles demonstrated 7-10-fold greater arterial levels of the biologically active agents compared to the unmodified nanoparticles in ex-vivo dog femoral, in vivo dog femoral, and pig coronary artery studies. See R. J. Levy et al, J. of Pharmaceutical Sciences, Vol. 87, No. 10, 1998, this reference and all references cited herein are incorporated herein in their entirety.

In one particular method, the method comprises inserting the drug delivery device (also referred to as a drug eluting device) as provided herein into a blood vessel. In one embodiment, the device is configured to provide at least an expandable portion that is a balloon or a scaffold or expandable-stent. As provided above, the balloon or scaffold typically has a long, narrow, hollow tube tabbed with a deflated balloon or contracted scaffold. The device is maneuvered through the cardiovascular system to the site of a blockage, occlusion requiring the selected biologically active agent or therapeutic agent. Once the balloon or scaffold is in the proper position, the balloon is inflated (or the scaffold expanded) and the outer surface of the balloon or scaffold contacts the internal walls of the blood vessel and/or a blockage or occlusion. The biologically active agent may be rapidly delivered to the target tissue by the reduction of the Au—S bond, releasing the substrate comprising the biologically active agent. In certain aspect, it is desired to deliver the agent to the tissue in as brief a period of time as possible while the device is deployed at the target site. The biologically active agent is released in the desired amount of time, usually in about 0.1 to 2 minutes, or about 0.1 to 1 minutes, while the balloon is inflated or while the scaffold is expanded and pressed against and in contact with the vessel wall. Once the delivery of the biologically active agent is completed for the desired amount for the selected period of time, the device may be removed from the site.

As will be appreciated by one of ordinary skill in the art, the drug-eluting scaffold or drug eluting balloon as exemplified in accordance with the present invention can be of any type. Any particular drug-eluting scaffold or drug eluting balloon described herein is for example purposes and not meant to be limiting of the invention.

1. A drug delivery device for the intraluminal controlled delivery of a biologically active agent comprising:
a dilating member comprising a proximal end and a distal end, and an inner surface and an outer surface, wherein a part of the outer surface of the dilating member is coated with a gold surface layer;
a biodegradable substrate comprising the biologically active agent, wherein the substrate is covalently bonded to the gold surface layer by a gold-sulfur (Au—S—) bond;
a first electrical lead having a first end and a second end, the first end connected to the gold surface layer, wherein the first electrical lead is configured to pass an electrical current to the gold surface layer; and
wherein the controlled delivery and release of the substrate comprising the biologically active agent is initiated by an electrochemical reduction and cleavage of the Au—S bond.

2. (canceled)

3. The drug delivery device of claim 1 for the intraluminal controlled delivery of a biologically active agent to an intraluminal surface, the drug delivery device further comprising:
an elongated insertion member having a proximal end and a distal end, wherein the dilating member is attached to the distal end of the elongated insertion member;
a second electrical load having a first end and a second end, the first end connected to a counter electrode, wherein the second electrical lead is configured to pass an electrical current to the counter electrode; and
wherein the controlled delivery and release of the substrate comprising the biologically active agent is initiated when the dilating member is directly contacting the intraluminal surface.

4. The device of claim 3, wherein the gold surface layer is placed only on the portion of the dilating member that in direct contact with the intraluminal surface when the dilating member is dilated;
at least a part of the counter electrode is placed on a portion that is not directly in contact with the intraluminal surface when the dilating member is dilated;
the second end of the first electrical lead is connected to an anode at the proximal side; and
the second end of the second electrical lead is connected to a cathode at the proximal side.

5. The device of claim 3 or 4, wherein the dilating member is a coronary scaffold or a balloon.

6. The device of claim 5, wherein the counter electrode main body on the balloon is placed on a proximal corn part of the balloon that does not directly contact the intraluminal surface when the balloon is dilated.

7-10. (canceled)

11. The device of claim 1, wherein the dilating member is a coronary scaffold or a balloon.

12. The device of claim 11, wherein the dilating member is a balloon and no portion of the gold surface layer exists on a folding line of the balloon.

13-14. (canceled)

15. The device of claim 11, further comprising a second electrical lead having a first end and a second end, the first end connected to a counter electrode.

16-18. (canceled)

19. The device of claim 5, wherein the coronary scaffold is made from a metal selected from the group consisting of stainless steel, platinum, titanium, tantalum, nickel-titanium, cobalt-chromium and their alloys thereof, or is made from a shape memory alloy or a superelastic alloy is selected from the group consisting of copper-zinc-aluminum-nickel, copper-aluminum-manganese, copper-aluminum-nickel and nickel-titanium alloy.

20. (canceled)

21. The device of any one of claims 1, 3, and 4, wherein the biodegradable substrate comprising a sulfur atom is covalently bonded to a hydrophobic fragment and a hydrophilic fragment, wherein the hydrophobic fragment comprises a biologically active agent; or wherein the biodegradable substrate comprising a sulfur atom is covalently bonded to a hydrophobic fragment that is bonded to a hydrophilic fragment that is further bonded to a hydrophobic fragment, wherein the hydrophobic fragment comprises a biologically active agent.

22. The device of claim 21, wherein the hydrophobic fragment further comprises a —C₅₋₁₈alkylenyl-linker- and the linker, is selected from the group consisting of —C(O)O—, —C(O)NH—, —OC(O)O—, —OC(S)O—, —OC(O)NH—, —NR¹C(O)O—, —SC(O)O—, —SC(O)S—, —NR¹C(NR¹)O— and —NR¹C(O)NR¹—, wherein each R¹ is independently H or C₁₋₃alkyl.

23. The device of claim 21, wherein the hydrophilic fragment comprises a biodegradable polymer selected from the group consisting of PAE, PCL, PLLA, PLA, PLGA, PHB, POE, polyketal, polyanhydride, polypeptide and PAE, and wherein the end group is selected from the group consisting of —OH, —NH₂, —C(O)OH, —NCO, —SH, biotin, and their block copolymer combinations thereof.

24. The device of claim 21, wherein the hydrophilic fragment comprises a biodegradable polymer that forms nanoparticles, nanogranulated particles, microparticles or microgranulated particles encapsulating the biologically active agent.

25. The device of claim 21, wherein the hydrophobic fragment and the hydrophilic fragment comprises —[—(C₅₋₁₈alkylenyl)ₘ-L-(CH₂CH₂O)ₙ—]ₚ—, wherein L is a linker selected from the group consisting of —C(O)O—, —C(O)NH—, —OC(O)O—, —OC(S)O—, —OC(O)NH—, —NR¹C(O)O—, —SC(O)O—, —SC(O)S—, —NR¹C(NR¹)O— and —NR¹C(O)NR¹—, wherein each R¹ is independently H or C₁₋₃alkyl, and where m is 1, 2 or 3, n is 1 to 90, and p is 1 to 10.

26. The device of any one of claims 1, 3, and 4, wherein the biologically active agent is selected from the group consisting of a carcinostatic, an immunosuppressive, an antihyperlipidemic, an ACE inhibitor, a calcium antagonist, an integrin inhibitor, an antiallergic, an antioxidant, a GPIIb/IIIa antagonist, retinoid, flavonoid, carotenoid, a lipid improvement agent, a DNA synthesis inhibitor, a tyrosine kinase inhibitor, an antiplatelet, a vascular smooth muscle antiproliferative agent, an anti-inflammatory agent, a biological material, an interferon and a NO production accelerator.

27-31. (canceled)

32. A method for the controlled delivery of a biologically active agent to an intraluminal surface using a drug delivery device, wherein the device comprises:
an elongated insertion member having a proximal end and a distal end;
a dilating member comprising a proximal end and a distal end, and an inner surface and an outer surface, wherein the proximal end of the dilating member is attached to the distal end of the elongated insertion member, and wherein a part of the surface of the dilating member is coated with a gold surface layer;
a biodegradable substrate comprising the biologically active agent, wherein the substrate is covalently bonded to the gold surface layer by a gold-sulfur (Au—S—) bond;
an electrical lead having a first end and a second end, the first end connected to the gold surface layer, wherein the electrical lead is configured to pass an electrical current to the gold surface layer; and
wherein the controlled delivery and release of the substrate comprising the biologically active agent is initiated by an electrochemical reduction and cleavage of the Au—S bond;
the method comprises inserting the device into the lumen and advancing the device until the dilating member is in a desired region of the intraluminal surface;
expanding the dilating member to contact the outer surface of the dilating member with the vessel wall; and
passing an electrical current to the electrical lead sufficient to reduce and cleave the Au—S bond and releasing the biodegradable substrate comprising the biologically active agent over a controlled time period.

33-38. (canceled)

39. The method of claim 32, wherein the biodegradable substrate comprising a sulfur atom is covalently bonded to a hydrophobic fragment and a hydrophilic fragment, wherein the hydrophobic fragment comprises a biologically active agent; or wherein the biodegradable substrate comprising a sulfur atom is covalently bonded to a hydrophobic fragment that is bonded to a hydrophilic fragment that is further bonded to a hydrophobic fragment, wherein the hydrophobic fragment comprises a biologically active agent, and the hydrophilic fragment comprises a biodegradable polymer that forms nanoparticles, nanogrannulated particles, microparticles or microgranulated particles encapsulating the biologically active agent.

40-41. (canceled)

42. A method of preparing a drug delivery device comprising a dilating member, with a substrate, the method comprising:
coating an outer surface of the dilating member in a dilated state with a layer of gold;
contacting the layer of gold with hydrophobic compound comprising a functional group and a thiol group, for a sufficient time to form a gold-sulfur (Au—S) bond between the hydrophobic compound and the layer of gold;
contacting the functional group of the hydrophobic compound with an activating group for a sufficient time to form an activated hydrophobic compound; and
contacting the activated hydrophobic compound with a hydrophilic polymer comprising a biologically active agent and an amine group to form the substrate.

43. (canceled)

44. The method of claim 42, wherein the coating of the outer surface of the dilating member is performed by dispensing, pipetting, ink jet deposit or chemical vapor deposition.

45. The method of claim 42, wherein the hydrophilic polymer comprising a biologically active agent forms a nano-granule, a micro-granule, a nanoparticle, or a microparticle.

46. (canceled)
